(19) United States
(12) Patent Application Publication
(10) Pub. No.: US 2025/0019235 A1
(43) Pub. Date: Jan. 16, 2025

(54) HYBRID-ELECTRIC PROCESS AND/OR SYSTEM FOR PRODUCING HYDROGEN

(71) Applicant: Iogen Corporation, Ottawa (CA)

(72) Inventors: Brian Foody, Ottawa (CA); Johannes H. J. Thijssen, Woodinville, WA (US); Patrick J. Foody, Ottawa (CA)

(21) Appl. No.: 18/711,991

(22) PCT Filed: Dec. 2, 2022
(86) PCT No.: PCT/CA2022/051768

§ 371 Date: May 21, 2024

Related U.S. Application Data
(60) Provisional application No. 63/368,812, filed on Jul. 19, 2022.
(60) Provisional application No. 63/264,923, filed on Dec. 3, 2021.

(51) Int. Cl. C01B 3/34; C01B 3/56; C12P 5/02

(52) U.S. Cl. CPC C01B 3/34; C01B 3/56; C12P 5/023; C01B 2203/0205; C01B 2203/0475; C01B 2203/068; C01B 2203/0833; C01B 2203/085; C01B 2203/1241

(57) ABSTRACT

A process and/or system for producing hydrogen or fuel, fuel intermediate, and/or chemical product produced using the hydrogen. The hydrogen is produced from a feedstock containing biomethane, where the biomethane is produced from biomass. The hydrogen production includes methane reforming a feed comprising biomethane, where at least part of the reforming is conducted in one or more electrically heated reactors (e.g., direct or indirect). Carbon-containing material derived from part of the biomass not converted to hydrogen is stored as part of one or more CCS processes.

### TECHNICAL FIELD

The present disclosure relates to a process and/or system for producing hydrogen, and in particular, relates to a process and/or system for producing hydrogen that combines the use of biomethane, low-carbon electricity for methane reforming, and carbon capture and storage.

### BACKGROUND

Fossil fuels like coal, oil, and natural gas supply a large percentage of the world's energy. Fossil fuels are also the primary human source of greenhouse gas (GHG) emissions. Concerns over climate change have imposed the need to reduce GHG emissions and/or reliance on fossil fuels. Many countries have made commitments to reach net-zero carbon emissions (i.e., decarbonization, where the economy either emits no GHG emissions or offsets its GHG emissions). Many believe electrification is the future (i.e., that fuels will be a thing of the past). As part of a decarbonization strategy, electrification typically involves replacing technology that runs on fossil fuel with technology that runs on electricity generated from low-carbon sources (e.g., nuclear power or renewable power such as hydro, solar, wind, geothermal, etc.). While electrification may play an important role in decarbonization, some sectors of the economy will remain hard or even impossible to electrify and thus may require other low-carbon alternatives. For example, long-haul trucking, shipping, aviation, some district heating, and/or energy-intensive industries (e.g., steel, cement, oil refining, and ammonia production) are often regarded as hard-to-decarbonize. Hydrogen and sustainable biofuels may play a key role in decarbonizing these hard-to-decarbonize sectors.

The role of hydrogen (H₂) with respect to decarbonization is increasingly discussed. Hydrogen is a versatile energy carrier with exceptional energy density. It can be used as a fuel, industrial feedstock (e.g., to produce fuel, fuel intermediates, or chemical products), or in fuel cells (e.g., to generate heat and/or electricity). In addition, hydrogen is already used in and/or can be used to displace fossil fuels, such as natural gas, in many of the hard-to-decarbonize sectors. However, while hydrogen produces zero emissions at its point of use, its carbon intensity (CI) can be highly dependent on how it is produced.

Most hydrogen produced today is produced from the processing of fossil fuels. Hydrogen produced from the steam methane reforming (SMR) of natural gas, which is often referred to as grey hydrogen, is generally associated with high GHG emissions (i.e., there is little to no climate benefits to using grey hydrogen). The GHG emissions from grey hydrogen production can be reduced by capturing and storing carbon dioxide (CO₂) produced from the SMR such that it is prevented from being released to the atmosphere (e.g., stored underground in suitable geological formations). Such carbon capture and storage (CCS), when combined with the SMR of natural gas, produces what is often referred to as blue hydrogen. Blue hydrogen can have a carbon intensity (CI) that is less than half that of grey hydrogen. However, blue hydrogen can still have significant lifecycle GHG emissions (e.g., fugitive methane emissions and/or emissions associated with the CCS process). Moreover, blue hydrogen is still reliant on fossil fuels. Concerns over climate change have imposed the need to reduce GHG emissions and/or reliance on fossil fuels.

Hydrogen can be produced from biomass (e.g., from the gasification or pyrolysis of biomass, or by reforming biogas produced by the anaerobic digestion of biomass). Hydrogen derived from biomass is referred to herein as “renewable hydrogen.”. The carbon intensity of renewable hydrogen can be highly dependent on the biomass and/or production process. In some cases, the carbon intensity of renewable hydrogen can be greater than that of blue hydrogen. As a fuel produced from biomass, renewable hydrogen is a biofuel, and thus a type of bioenergy. The supply of biomass, and in particular of biogas, is often viewed as limited, thereby bringing into question its potential to significantly contribute to decarbonization. Biogas is itself a biofuel, the highest and best use of which is often considered to be simple combustion. Combusting biogas can produce low-carbon electricity, which can be used in the electrified sectors.

Hydrogen also can be produced from the electrolysis of water (e.g., using electricity generated from low-carbon sources to split water into hydrogen and oxygen (O₂) in a unit referred to as an electrolyser). Hydrogen produced by the electrolysis of water using renewable electric power is referred to herein as “green hydrogen.” Green hydrogen can have a carbon intensity close to zero and has been considered by some as the only zero-carbon option for hydrogen production. Green hydrogen is often viewed as having a critical role in decarbonization. The supply potential of green hydrogen may be linked to the supply potential of solar and wind power, and thus is often viewed as sufficient to exceed global energy demand. In addition to potentially being able to produce enough hydrogen to help decarbonize the hard-to-electrify sectors (e.g., replace fossil fuels as a low-carbon feedstock in chemicals and/or fuel production). In theory, green hydrogen also has the potential to store surplus renewable power when the electricity grid cannot absorb it. However, while green hydrogen is often viewed as playing a critical role in decarbonation, it currently is energy intensive and costly (e.g., green hydrogen may cost, on average, between two and three times more to make than blue hydrogen). In general, these costs may be related to the high capital associated with electrolysers and/or the cost of renewable electricity (e.g., relative to natural gas). While falling renewable electricity costs and improvements in electrolyser technology may make green hydrogen more competitive with blue hydrogen, a relatively low electrical efficiency (e.g., 60% to 80%) means that an excessive amount of renewable electricity is required and/or an excessive amount of heat is lost in the process.

### SUMMARY

The present disclosure relates generally to process(es) and/or system(s) that facilitate producing hydrogen more efficiently from scarce and/or expensive resources and/or facilitate hydrogen production that can decrease global GHG emissions (e.g., produce hydrogen having a low and/or negative carbon intensity). The process(es) and/or system(s) may be related to the production of hydrogen, or may be related to producing a fuel, fuel intermediate, chemical product, or any combination thereof using the hydrogen.

One approach for more efficient hydrogen production includes: a) using feedstock comprising biomethane, and thus at least partially derived from biomass, for the hydrogen production process, and b) using low-carbon electricity such as renewable electricity to provide heat for the hydrogen production process, and in particular for the methane reforming. When a process produces hydrogen that is both: (i) at least partially derived from biomass, and (ii) produced using methane reforming wherein the reforming heat is generated by electricity (i.e., directly or indirectly), the process is referred to herein as a hybrid-electric process. The process(es) and/or system(s) disclosed herein (e.g., hybrid-electric process(es) and/or system(s)) are particularly efficient when the feedstock comprises biomethane, the biomethane is produced from biomass from different sources, the methane reforming is SMR (i.e., endothermic), and/or energy in the syngas produced from the methane reforming is at least about 110%, at least about 115%, or at least about 120%, of the energy of the feedstock subjected to methane reforming, for a given time period (e.g., maximum may be about 129%). Providing this type of uplift (e.g., of at least 10%) requires a certain extent of reformer electrification that may push the efficiency into territory where the overall efficiency of the process is relatively high. Using low-carbon electricity to provide this thermal lift can reduce the GHG emissions of the process (i.e., relative a similar SMR that uses natural gas to fire a top or side fired furnace).

One approach proposed for more efficient hydrogen production and/or to produce hydrogen having a low and/or negative carbon intensity is to combine: a) using feedstock comprising biomethane, and thus at least partially derived from biomass, for the hydrogen production process, b) using low-carbon electricity such as renewable electricity to provide heat for the hydrogen production process, and in particular for the methane reforming, and c) storing carbon-containing material derived from the biomass as part of one or more CCS processes. It has now been found that combining the use of low-carbon electricity, bioenergy, and CCS into an integrated hybrid-electric process that uses biomethane feedstock may have surprising and compelling advantages over the production of green hydrogen, including a higher energy efficiency and greater atmospheric carbon dioxide removal, for a given quantity (e.g., MJ) of hydrogen produced. Moreover, a rough analysis of the economics suggests that the hybrid-electric approach may be comparable to green hydrogen production, particularly when credits for CCS are accounted for.

One approach for producing hydrogen having a low and/or negative carbon intensity is to combine: a) using feedstock comprising biomethane, and thus at least partially derived from biomass, for the hydrogen production process where the biomethane is produced from the anaerobic digestion of biomass, and b) storing carbon-containing material derived from the biomass, wherein at least two types of carbon-containing materials are stored and/or carbon-containing material produced in different parts of the process are stored (e.g., multiple CCS processes). In particular, it can be advantageous to provide one or more CCS processes (e.g., three) that include storage of carbon dioxide produced from the biomethane production process, storage of carbon dioxide produced from the hydrogen production process, and storage of carbon-containing material derived from digestate produced from the anaerobic digestion. It has now been found that providing such three-tiered CCS process(es) can significantly reduce the carbon intensity of hydrogen produced from biogas and/or of a fuel, fuel intermediate, or chemical product, produced from the hydrogen. Moreover, when integrated with the hybrid-electric approach using low-carbon electricity, this three-tiered CCS approach may deliver the scale needed to meet global net-zero aspirations with relatively high energy efficiency (e.g., relative to green hydrogen production).

In accordance with one aspect of the instant invention there is provided a process of producing fuel, fuel intermediate, chemical product, or any combination thereof, the process comprising: providing biomethane, the biomethane produced from a biomethane production process comprising converting biomass to biomethane; generating hydrogen in a hydrogen production process comprising: (a) producing syngas in a process comprising subjecting a feed comprising the biomethane to methane reforming, the methane reforming conducted in one or more reactors, at least one of the one or more reactors configured to provide electrically generated heat for the methane reforming, the heat for the methane reforming at least partially produced using low-carbon electricity, and, (b) subjecting the syngas to a purification process wherein hydrogen is separated from at least carbon dioxide, thereby producing a stream enriched in hydrogen; wherein carbon-containing material derived from the biomass is stored and/or used in at least one carbon capture and storage process, wherein the carbon-containing material comprises: (i) carbon dioxide produced from the hydrogen production process, and (ii) carbon dioxide produced from the biomethane production process; and wherein inputs, outputs, or a combination thereof from each of the biomethane production process, the hydrogen production process, and the at least one carbon capture and storage process are monitored, wherein each of the inputs is a material input or energy input and wherein each of the outputs is a material output or an energy output.

In accordance with one aspect of the instant invention there is provided a process of producing hydrogen, the process comprising: providing biomethane, the biomethane produced from a biomethane production process comprising anaerobic digestion of biomass, the anaerobic digestion producing biogas and digestate; generating hydrogen in a hydrogen production process comprising: (a) producing syngas in a process comprising subjecting a feed comprising the biomethane to methane reforming, the methane reforming conducted in one or more reactors, at least one of the one or more reactors configured to provide electrically generated heat for the methane reforming, the heat for the methane reforming at least partially produced using low-carbon electricity, and, (b) subjecting the syngas to a purification process wherein hydrogen is separated from at least carbon dioxide, thereby producing a stream enriched in hydrogen; wherein carbon-containing material derived from the biomass is stored and/or used in at least one carbon capture and storage process, wherein the carbon-containing material comprises: (i) carbon dioxide produced from the hydrogen production process, (ii) carbon dioxide from the biogas, and (iii) carbon-containing material produced from processing at least part of the digestate.

In accordance with one aspect of the instant invention there is provided a process of producing hydrogen, the process comprising: providing biomethane, the biomethane produced from a biomethane production process comprising anaerobic digestion of biomass, the anaerobic digestion producing biogas and digestate, the biomethane used in a hydrogen production process comprising: (a) producing syngas in a process comprising subjecting a feed comprising the biomethane to methane reforming, the methane reforming conducted in one or more reactors, at least one of the one or more reactors configured to provide electrically generated heat for the methane reforming, the heat for the methane reforming at least partially produced using low-carbon electricity, and, (b) subjecting the syngas to a purification process wherein hydrogen is separated from at least carbon dioxide, thereby producing a stream enriched in hydrogen; wherein carbon-containing material derived from the biomass is stored and/or used in at least one carbon capture and storage process, wherein the carbon-containing material comprises: (i) carbon dioxide produced from the hydrogen production process, (ii) carbon dioxide from the biogas, and (iii) carbon-containing material produced from processing at least part of the digestate.

### BRIEF DESCRIPTION OF THE DRAWINGS

Further features and advantages of the present disclosure will become apparent from the following detailed description, taken in combination with the appended drawings, in which:

FIG. 1 is a process flow diagram in block form in accordance with an embodiment of the instant disclosure;

FIG. 2 is a process flow diagram in block form in accordance with an embodiment of the instant disclosure, wherein the biomethane production comprises anaerobic digestion;

FIG. 3 is a process flow diagram in block form in accordance with an embodiment of the instant disclosure, wherein the biomethane production comprises multiple anaerobic digestions;

FIG. 4 is a process flow diagram in block form in accordance with an embodiment of the instant disclosure, wherein the biomethane production comprises gasification;

FIG. 5a is a schematic diagram of a SMR wherein heat for reforming is provided from the combustion of methane-containing gas;

FIG. 5b is a schematic diagram of a SMR wherein heat for reforming is provided using low-carbon electricity directly;

FIG. 5c is a schematic diagram of a SMR wherein heat for reforming is provided using low-carbon electricity indirectly;

FIG. 6a is process flow diagram in block form of a hydrogen production process that includes SMR wherein the heat for reforming is provided by combusting methane-containing gas;

FIG. 6b is a process flow diagram in block form of a hydrogen production process that includes SMR wherein the heat for reforming is provided by combusting methane-containing fuel gas and tail gas from hydrogen purification;

FIG. 6c is a process flow diagram in block form of a hydrogen production process that includes SMR wherein the heat for reforming is provided by low-carbon electricity;

FIG. 7a is a process flow diagram in block form of a process for producing hydrogen according to base case 1 (i.e., conventional SMR without CCS);

FIG. 7b is a process flow diagram in block form of a process for producing hydrogen according to base case 2 (i.e., conventional SMR with CCS);

FIG. 7c is a process flow diagram in block form of a process for producing hydrogen according to Example 1 (i.e., hybrid-electric SMR with CCS); and

FIG. 7d is a process flow diagram in block form of a process for producing hydrogen according to Example 3.

It will be noted that throughout the appended drawings, like features are identified by like reference numerals.

### DETAILED DESCRIPTION

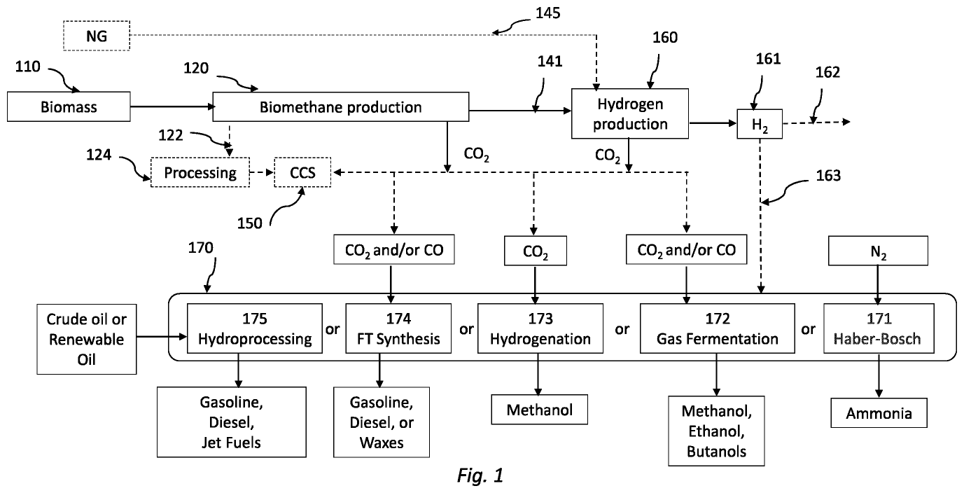

Referring to FIGS. 1 through 4, there is shown illustrative process(es) and/or system(s) according to certain embodiments of the instant disclosure. Prior to describing these figures in more detail, it is to be noted that the figures are simplified flow diagrams and do not necessarily reflect all of the steps/units/equipment that may be incorporated. The incorporation of such steps/units/equipment is well known and will be understood by those skilled in the art.

### Biomass

The process(es) and/or system(s) of the instant disclosure produce and/or use biomethane derived from biomass. Biomass refers to organic material originating from plants, animals, or micro-organisms (e.g., including plants, agricultural crops or residues, municipal wastes, animal wastes, and algae). Biomass is a renewable resource, which can be naturally replenished on a human timescale, and which can be used to produce bioenergy and/or biofuels (e.g., biogas). In general, the biomass 110 can be any suitable biomass (e.g., one or more types of biomass feedstock). Some examples of suitable biomass may include: (i) energy crops (e.g., switchgrass, sorghum, etc.); (ii) residues, byproducts, or waste from the processing of plant material in a facility, or feedstock derived therefrom (e.g., sugarcane bagasse, sugarcane tops/leaves, corn stover, etc.); (iii) agricultural residues (e.g., wheat straw, corn cobs, barley straw, corn stover, etc.); (iv) forestry material; (v) livestock manure, including sheep, swine, and cow manure; (vi) food scraps and/or agrifood processing residues (e.g., from slaughterhouse), and/or (vii) municipal waste or components removed or derived from municipal waste. These examples of suitable biomass are advantageous in that they do not compete with food production. The use of forestry or agricultural feedstocks (e.g., energy crops, residues, byproducts, or waste from the processing of plant material in a facility, or feedstock derived therefrom, or agricultural residues) may be advantageous for reducing GHG emissions. The use of livestock manure, such as swine or cow manure, may be advantageous in terms of reducing the lifecycle GHG emissions the hydrogen, or fuel, fuel intermediate, or product (e.g., chemical product) produced using the hydrogen. The use of fibrous biomass (e.g., bagasse, coconut husk, straw, reed, alfalfa, etc.) and/or biomass having fibrous component, may be advantageous in terms of having the potential to increase the supply of biogas. For example, while the supply of biogas from landfills and/or manure is substantially limited, the use of fibrous biomass to produce biogas has the potential to increase supply. Advantageously, the supply can be increased using agricultural residues.

### Biomethane Production

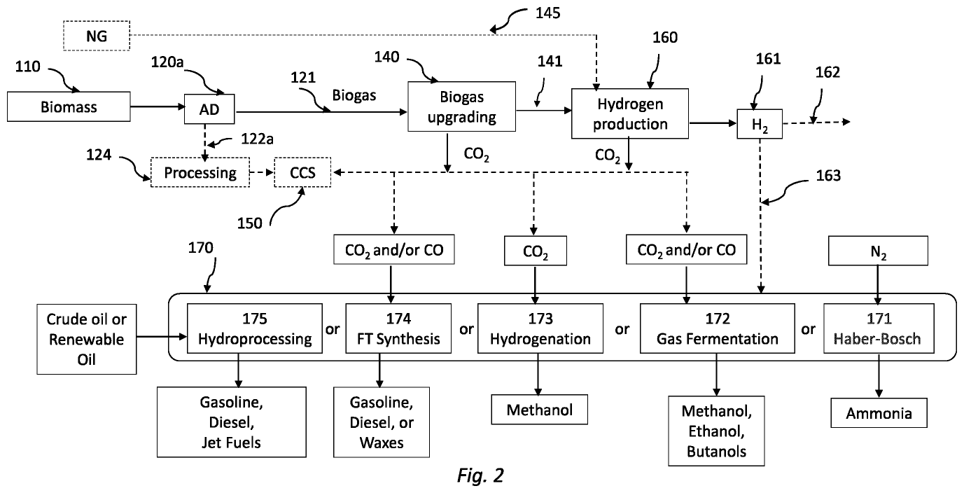

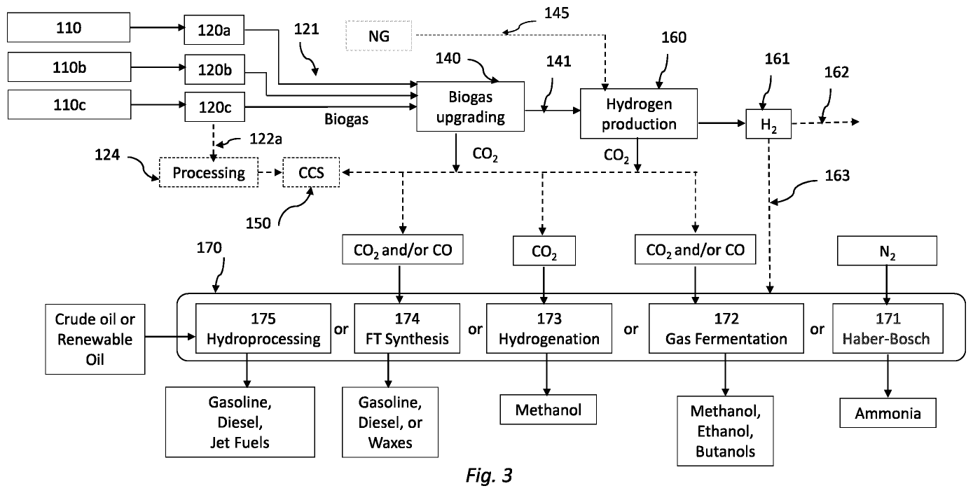

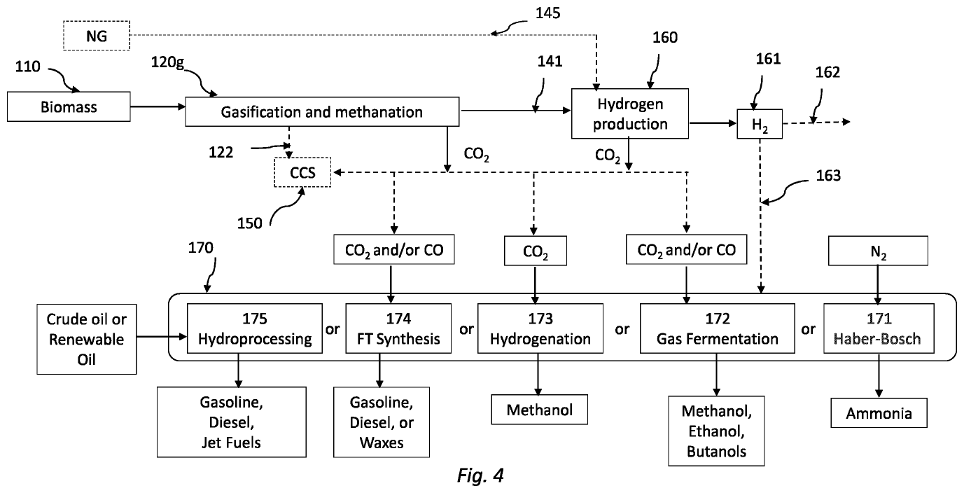

In general, at least part of the biomass is converted to upgraded biogas (e.g., biomethane) in biomethane production. Biomethane production 120 can include any suitable process or combination of processes that can convert at least part of the biomass to upgraded biogas (e.g., biomethane). For example, the biomethane production process can include anaerobic digestion 120a, which produces biogas, and biogas upgrading 140 as illustrated in FIGS. 2 and 3. The term “biogas”, as used herein, refers to a gas mixture that contains methane produced from biomass. Alternatively, the biomethane production process can include gasification and methanation 120 g as illustrated in FIG. 4, or pyrolysis (not shown). In addition to producing upgraded biogas (e.g., biomethane), the biomethane production may produce residue (e.g., carbon-containing material 122) that is not converted to biogas/biomethane. Optionally, this residue, or a carbon-containing material produced by processing 124 (e.g., combusting) at least part of this residue is provided and/or stored as part of one or more carbon-capture and storage processes (e.g., 150 in FIGS.). The phrase “derived from biomass”, with reference to upgraded biogas, biomethane, or carbon-containing material, means that the upgraded biogas, biomethane, or carbon-containing material, respectively, is produced from the biomass from one or more processes (e.g., directly or indirectly). The phrase “derived from residue”, with reference to carbon-containing material, means that the carbon-containing material is produced from the residue (e.g., liquid and/or solid) from one or more processes (e.g., directly or indirectly).

Referring to the embodiment in FIG. 2, at least part of the biomass is subjected to anaerobic digestion 120a. Anaerobic digestion refers to the biological breakdown of organic matter by anaerobic microorganisms, is typically conducted in anaerobic or low oxygen conditions, and may involve a series of microorganism types and processes (e.g., hydrolysis, acidogenesis, acetogenesis, and methanogenesis). In general, the anaerobic digestion of biomass can be conducted in any suitable environment, including a natural environment (e.g., a landfill) or a controlled environment (e.g., one or more anaerobic digesters arranged in series and/or in parallel). Each anaerobic digester can be a holding tank, or another contained volume, such as a covered lagoon or sealed structure, configured to facilitate the anaerobic digestion and collection of biogas. For example, each anaerobic digester can be a plug flow system or basin type reactor. Such anaerobic digesters can be single-stage or multi-stage digester systems and/or may be designed and/or operated in a number of configurations including batch or continuous, mesophilic or thermophilic temperature ranges, mixed or unmixed, and low, medium, or high rates. The anaerobic digestion conducted in such digesters can use a nutrient solution, which may improve the conversion, particularly for fibrous biomass. Using a controlled environment facilitates monitoring input and output material flows, which can be used to determine how much biogas is produced from the anaerobic digestion of a certain amount of biomass, and/or which can be used to calculate lifecycle GHG emissions and/or validate compliance (e.g., with a pathway).

In general, the feedstock for anaerobic digestion 120a can be any suitable biomass. For example, it can be raw or pretreated biomass, or can be biomass that is produced from another process (e.g., can be waste, residue, and/or byproduct from another process). In certain embodiments, the biomass includes a fibrous feedstock (e.g., straw) and/or an agricultural residue. In such embodiments, the biomass may be received as bales or may be baled. In certain embodiments, the biomass is subjected to anaerobic digestion as substantially intact bales or is unbaled prior to anaerobic digestion. The biomass (e.g., baled or unbaled) may be pretreated prior to anaerobic digestion. Such pretreatment can include size reduction, sand removal, slurry formation, the addition of chemicals and/or heat (e.g., steam explosion), and/or nutrients provided for anaerobic digestion. Advantageously, size reduction can accelerate the anaerobic digestion process and/or improve material handling. Some examples of size reduction methods include milling, grinding, agitation, shredding, compression/expansion, and/or other types of mechanical action. Size reduction by mechanical action may be performed by any type of equipment adapted for the purpose, for example, but not limited to, hammer mills, tub-grinders, roll presses, refiners, hydropulpers, and hydrapulpers. In certain embodiments, biomass having an average particle size that is greater than about 6-8 inches is subject to a size reduction wherein at least 90% by volume of the particles produced from the size reduction have a length between about 1/16 inch and about 6 inches.

In certain embodiments, the biomass fed to anaerobic digestion includes waste and/or residue from another process (e.g., ethanol production). Ethanol production, may for example, produce corn ethanol, sugar ethanol, or cellulosic ethanol, in addition to carbon dioxide. The waste and/or residue can include aqueous streams (e.g., condensate streams), solids filtered from one or more streams (e.g., after hydrolysis), and/or at least part of the still bottoms (e.g., from ethanol recovery). In certain embodiments, carbon dioxide from ethanol production is provided as part of the carbon capture and storage processes.

The biogas 121 produced by the anaerobic digestion of biomass is a gas mixture that typically contains methane (CH₄) and carbon dioxide (CO₂), and that may contain water (H₂O), nitrogen (N₂), hydrogen sulfide (H₂S), ammonia (NH₃), oxygen (O₂), volatile organic compounds (VOCs), and/or siloxanes, depending on the biomass from which it is produced. Biogas produced from anaerobic digestion often has a methane content between about 35% and 75% (e.g., about 60%) and a carbon dioxide content between about 15% and 65% (e.g., about 35%). The percentages used to quantify gas composition and/or a specific gas content, as used herein, are expressed as mol %, unless otherwise specified. More specifically, they are expressed by mole fraction at standard temperature and pressure (STP), which is equivalent to volume fraction.

When conducted in one or more anaerobic digesters, the anaerobic digestion of biomass also produces a potentially usable digestate 122a. Digestate refers to the liquid and/or solid material remaining after one or more stages of anaerobic digestion (e.g., may refer to acidogenic digestate, methanogenic digestate, or a combination thereof). Digestate can include organic material not digested by the anaerobic microorganisms (e.g., fibrous undigested organic material made of lignin and cellulose), byproducts of the anaerobic digestion released by the microorganisms, and/or the microorganisms themselves. For example, the digestate can include carbohydrates, nutrients (such as nitrogen compounds and phosphates), other organics, and/or wild yeasts. The composition of digestate can vary depending on the biomass from which it is derived. Digestate often has both a solid and liquid component. One use of digestate is as a soil conditioner, where it can provide nutrients for plant growth and/or displace the use of fossil-based fertilizers. However, as a soil conditioner, digestate may have a significant methane formation potential, and thus may be associated with GHG emissions. In certain embodiments of the instant disclosure, at least part of the digestate is processed 124 (e.g., combusted, or subjected to gasification, pyrolysis, hydrothermal treatment, and/or wet oxidation) to provide carbon-containing material for CCS.

The biogas produced in anaerobic digestion is subjected to biogas upgrading. Biogas upgrading refers to a process where biogas (e.g., raw or cleaned biogas) is treated to remove one or more components (e.g., CO₂, N₂, H₂O, H₂S, O₂, NH₃, VOCs, siloxanes, and/or particulates), wherein the treatment increases the calorific value of the biogas. For example, biogas upgrading typically includes removing carbon dioxide and/or nitrogen. In general, biogas upgrading can be conducted using any suitable technology or combination of technologies known in the art. Biogas upgrading, which is well-known, often includes one or more of the following technologies: 1) absorption, 2) adsorption, 3) membrane separations, and 4) cryogenic upgrading. As will be understood by those skilled in the art, the technology or combination of technologies selected may be dependent on the composition of the biogas and/or how it is produced. Since biogas often has a significant carbon dioxide content, biogas upgrading plants often include at least one system for separating methane from carbon dioxide. Some examples of technologies that can remove carbon dioxide from biogas include, but are not limited to, absorption (e.g., water scrubbing, organic physical scrubbing, chemical scrubbing (e.g., amine)), adsorption (e.g., pressure swing adsorption (PSA), which includes vacuum PSA, or temperature swing adsorption), membrane separation (e.g., CO₂ selective membranes based on polyimide, polysulfone, cellulose acetate, polydimethylsiloxane), and cryogenic separation. Optionally, biogas upgrading can include increasing the calorific value of the biogas by adding gas having a relatively high energy content (e.g., propane, natural gas, liquified petroleum gas (LPG)).

Preferably, the biogas upgrading 140 produces biomethane. When produced from biogas upgrading, biomethane refers to: (1) biogas that has been upgraded to meet or exceed applicable natural gas distribution system specifications (e.g., pipeline specifications), (2) biogas that has been upgraded to meet or exceed applicable quality specifications for vehicle use (e.g., CNG specifications), and/or (3) natural gas withdrawn from a natural gas distribution system that is associated with the environmental attributes of upgraded biogas injected into the natural gas distribution system (e.g., a gas that qualifies as biomethane under applicable regulations). With respect to (1), pipeline specifications, which can include specifications required for biogas for injection into a natural gas distribution system, may vary by region and/or country in terms of value and units. For example, pipelines standards may require the biomethane to have a CH₄ content that is at least 95% or have a heating value of at least 950 BTU/scf. With respect to (3), since the transfer or allocation of the environmental attributes of the upgraded biogas injected into the natural gas distribution system to gas withdrawn at a different location is typically recognized, the withdrawn gas is recognized as biomethane and/or qualifies as biomethane under applicable regulations (e.g., even though the withdrawn gas may not contain actual molecules from the original biomass and/or contains methane from fossil sources). Such transfer may be carried out on a displacement basis, where transactions within the natural gas distribution system involve matching and balancing of inputs and outputs. Typically, the direction of the physical flow of gas is not considered. The term “environmental attributes”, as used herein with regard to a specific material (e.g., biomethane), refers to any and all attributes related to the material, including all rights, credits, benefits, or payments associated with the renewable nature of the material and/or the reduction in or avoidance of fossil fuel consumption or reduction in lifecycle GHG gas emissions associated with the use of the material. Some non-limiting examples of environmental attributes include verified emission reductions, voluntary emission reductions, offsets, allowances, credits, avoided compliance costs, emission rights and authorizations, certificates, voluntary carbon units, under any law or regulation, or any emission reduction registry, trading system, or reporting or reduction program for GHG gas emissions that is established, certified, maintained, or recognized by any international, governmental, or nongovernmental agency.

In general, the biogas upgrading can be conducted at one or more biogas upgrading facilities. For example, in certain embodiments the biogas 121 provided for biogas upgrading 140 includes multiple biogases, where each biogas is produced from a different anaerobic digestion 120a, 120b, 120c, which can have different biomass feedstocks 110, 110b, 110c, as illustrated in FIG. 3. In this case, the biogas upgrading can be conducted at a plurality of biogas upgrading facilities (e.g., decentralized facilities, not shown), each biogas upgrading facility being in close proximity to one of the anaerobic digestions 120a, 120b, 120c. Alternatively, or additionally, the biogas upgrading can be conducted at a centralized biogas upgrading facility that receives raw or partially purified biogases produced from the different anaerobic digestions 120a, 120b, 120c. When the biogas upgrading produces biomethane, the biomethane produced can be transported using a natural gas distribution system (if required). Accordingly, the biomethane can be transported cost effectively and/or from a relatively large geographical area. Providing biomethane produced from biomass from different sources (e.g., different farms), and in particular from different biomethane producers, can be advantageous for providing biomethane having a carbon intensity below a certain value and/or to increase scale of the process.

Referring to the embodiment in FIG. 4, the biomass is subjected to gasification followed by methanation 120 g. Gasification refers to a process that converts biomass and/or fossil-based carbonaceous materials at high temperatures (e.g., >700° C.), without combustion, with a controlled amount of oxygen and/or steam into gas mixture primarily composed of carbon monoxide (CO) and hydrogen and sometimes carbon dioxide, referred to as syngas. For example, the syngas produced by the gasification of wood may include carbon monoxide, carbon dioxide, hydrogen, methane, ethylene (C₂H₄), ethane (C₂H₆), dust (ash), tar, chloride, sulfur, etc. Following gasification, the syngas is often subjected to cooling, tar removal, and/or cleaning. The syngas may then be subjected to methanation, a catalytic conversion wherein carbon dioxide and carbon monoxide in the syngas can undergo the following reactions:

CO+3H₂→CH₄+H₂O (1)

CO₂+4H₂→CH₄+2H₂O (2)

Methanation, which is well-known in the art, typically is carried out in the presence of a solid catalysis (e.g., nickel-based catalyst). The gas produced by this gasification and methanation approach typically contains methane (and possibly ethane) and water, and can include carbon dioxide. The gas can be purified and/or dried to provide biomethane. Methanation units, which can include a water gas shift reactor, a carbon dioxide scrubber, a methanation reactor, and a dehydration system, are often configured to produce biomethane. When produced from gasification of biomass followed by methanation, biomethane refers to: (1) a near-pure source of methane derived from the biomass that can meet or exceed applicable natural gas distribution system specifications (e.g., pipeline specifications), (2) a near-pure source of methane derived from the biomass that can meet or exceed applicable quality specifications for vehicle use (e.g., CNG specifications), and/or (3) natural gas withdrawn from a natural gas distribution system that is associated with the environmental attributes of a near-pure source of methane derived from the biomass and injected into the natural gas distribution system (e.g., a gas that qualifies as biomethane under applicable regulations). A possible byproduct of biomass gasification is biochar (biological charcoal). Carbon-containing material not converted to biomethane 122 (e.g., residue such as biochar) and/or carbon dioxide produced from gasification may be provided as part of a CCS 150.

### Hydrogen Production

In general, the upgraded biogas (e.g., biomethane) is subjected to hydrogen production. In general, the hydrogen production 160 can use any suitable technology known in the art that can convert methane-containing gas such as biomethane and/or natural gas to hydrogen. Examples of technologies that may be suitable include, but are not limited to, steam methane reforming (SMR), autothermal reforming (ATR), partial oxidation (POX), and dry methane reforming (DMR). SMR, ATR, and DMR, which are types of catalytic reforming, may operate by exposing natural gas to a catalyst at high temperature and pressure to produce syngas. POX reactions, which include thermal partial oxidation reactions (TPOX) and catalytic partial oxidation reactions (CPOX), may occur when a sub-stoichiometric fuel-oxygen mixture is partially combusted in a reformer. POX also may be referred to as oxidative reforming. For purposes herein, the term “methane reforming” may refer to SMR, ATR, DMR, or POX. Methane reforming is well known in art. Of the various types of methane reforming, SMR is the most common.

In certain embodiments, the hydrogen production includes SMR. In SMR, which is an endothermic process, methane is reacted with steam under pressure in the presence of a catalyst to produce carbon monoxide (CO) and H₂ according to the following reaction:

CH₄+H₂O+heat→CO+3H₂ (3)

The SMR reaction may occur in the SMR reactor tubes, which contain the reforming catalyst. Without being limiting, the catalyst may be nickel-based, the operating pressure may be between 200 psig (1.38 MPa) and 600 psig (4.14 MPa), and the operating temperature may be between about 450 to 1000° C.

In certain embodiments, the hydrogen production includes DMR. In DMR, methane reacts with carbon dioxide, rather than water, according to the following reaction:

CO₂+CH₄→2CO+2H₂ (4)

Without being limiting, the DMR catalyst may be nickel, iron, ruthenium, palladium, or platinum based. While the DMR process does not require steam, and may be conducted at lower temperatures, it may be limited by the potential for coke formation.

In certain embodiments, the hydrogen production includes ATR. ATR combines partial oxidation and catalytic steam or carbon dioxide reforming of methane in a single reactor. Heat generated from the partial oxidation (e.g., in the combustion zone of the reactor) may be used in the catalytic reforming (e.g., in the reforming zone of the reactor). Accordingly, a common stand-alone ATR may not require the supply or dissipation of thermal energy. The ATR reactions include:

2CH₄+O₂+CO₂→3H₂+3CO+H₂O (5)

4CH₄+O₂+2H₂O→10H₂+4CO (6)

The syngas produced from methane reforming (e.g., Eqs. 3, 4, 5, or 6) may be further reacted in a water gas shift (WGS) reaction, wherein carbon monoxide is converted to carbon dioxide and hydrogen:

CO+H₂O→CO₂+H₂+small amount of heat (7)

Although optional, providing WGS downstream of methane reforming increases the yield of H₂, and thus is commonly included in hydrogen production. When included, the WGS is considered to be part of the methane reforming. The syngas produced from methane reforming often includes hydrogen, methane, carbon monoxide, carbon dioxide and water vapour. As will be understood by those skilled in the art, methane reforming can be conducted using one or more reactors. For example, the WGS can be conducted using a high temperature WGS reactor followed by a low temperature WGS reactor.

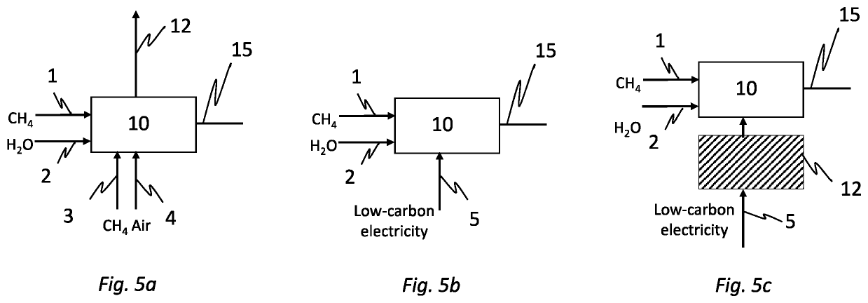

In certain embodiments, heat required for the catalytic reforming is provided by combustion of methane containing gas in the reformer burners (e.g., a combustion chamber may surround the reformer tubes that contains the catalyst and in which the reforming reaction is conducted). For example, consider the SMR illustrated in FIG. 5a. Methane-containing feedstock is provided for hydrogen production. A portion of this feedstock is preheated and is fed as feed stream 1, along with steam 2, into the reactor tubes for the methane reforming 10, which contain the reforming catalyst. Another portion is provided as fuel gas 3 (e.g., natural gas), which is fed with combustion air 4 into the reformer burners, which provide heat (e.g., required for an endothermic reforming reaction). The syngas 15 produced from the methane reforming may be fed to WGS (not shown) to produce more hydrogen. In such embodiments, the feed 1 can include natural gas in addition to upgraded biogas. The reformers may be characterized by the location of the burners within the combustion chamber (e.g., side-fired, top-fired, bottom-fired). Such fired burners are commonly used in hydrogen production. Unfortunately, the use of fired burners produces a flue gas that contains a significant amount of carbon dioxide. As result of low pressure and/or low carbon dioxide concentration, carbon dioxide in the flue gas can be challenging (e.g., economically) to remove and/or isolate for CCS purposes. In general, the upgraded biogas can be provided as feed for the methane reforming, as fuel for the methane reforming, or used as both feed and fuel for the methane reforming. In certain embodiments of the instant disclosure, the fuel 3 for the methane reforming contains biomethane. Since combusting biomethane simply returns to the atmosphere carbon that was recently fixed by photosynthesis, and thus is considered relatively benign, this can reduce GHG emissions from the SMR furnace (e.g., compared to using fossil-based methane).

In general, heat required for the methane reforming (e.g., catalytic reforming) is generated using low-carbon electricity (e.g., at least in part). Low-carbon electricity refers to electricity generated in a process that does not emit significant amounts of fossil-based carbon dioxide and/or is produced from renewable energy sources. Without being limiting, low-carbon electricity can include electricity produced using nuclear power, hydropower, solar power, wind power, geothermal power, wave power, tidal power, or electricity produced from the combustion of a low-carbon energy source (e.g., biomass, biogenic syngas, or hydrogen) or of a fossil-based energy source with CCS. In certain embodiments, heat required for the catalytic reforming is generated using renewable electricity (i.e., electricity produced using renewable energy sources such as hydropower, solar power, wind power, geothermal power, wave power, tidal power, etc.). In certain embodiments, the low-carbon electricity is generated from gasification of agricultural and/or solid waste. In certain embodiments, heat required for the methane reforming (e.g., catalytic reforming) is generated using low-carbon electricity and the from the combustion of methane contain gas (e.g., non-renewable and/or renewable methane containing gas). For example, in certain embodiments, the methane reforming includes at least one methane reformer heated using low carbon electricity and at least one methane reformer heated using fuel gas (e.g., renewable and/or no-renewable).

In general, any suitable technology known in the art that can use electricity to produce a sufficient heat for the methane reforming can be used. The low-carbon electricity can produce the heat for methane reforming directly (e.g., FIG. 5b) and/or indirectly (FIG. 5c). Comparing FIG. 5a to FIGS. 5b and 5c, the low-carbon electricity 5, or combination of low-carbon electricity and a heat transfer medium and/or heat storage fluid 12, can replace the conventional methane co-firing, and thus can eliminate the use of the fuel 3 and the production of flue gas 12. Since there is no flue gas, the electrically heated methane reforming may have carbon emissions that are reduced by 20-50% relative to the conventional natural gas/methane co-firing of FIG. 5a. Advantageously, these reduced carbon emissions are achieved without relying on a more economically challenging step of capturing carbon dioxide from the flue gas.

For illustrative purposes, the methane reforming in FIGS. 5a, 5b, and 5c is shown as SMR. Those skilled in the art will understand that these methods of providing heat for the reforming also can be used for other methane reforming technologies, including ATR and/or DMR. It may be particularly advantageous to use electrically-heated reformers that include ATR (e.g., as the lower temperatures may be more compatible with the use of heat storage mediums) or DMR (e.g., as the electrically-heated reformers may be less susceptible to coke formation). In certain embodiments, the low-carbon electricity is used to provide radiant heat or conductive heat.

In certain embodiments, the low-carbon electricity is used to provide heat for methane reforming directly (e.g., to power resistive or inductive heaters that provide the heat directly for the methane reforming). In such embodiments, the low-carbon electricity may be at least partially provided from a battery system.

In certain embodiments, heat for the catalytic reforming is provided by direct electrical resistance. For example, the reformer can include a stainless steel tube, the inside of which is coated with a thin layer of catalyst (e.g., nickel). In this case, each end of the tube can be coupled to a power generator such that the tube provides the electrical resistance. Alternatively, the reformer can include a tube containing the electric resistance, where the electric resistance is coated with a thin layer of catalyst (e.g., nickel). Further alternatively, the reactor can include a tube that includes a macroscopic structure (e.g., that provides channels for fluid flow) that is coated with ceramic coating impregnated with a catalytically active material. Such designs may provide uniform heat distribution and/or compact footprints (e.g., because the furnace, which can be large, is not required).

In certain embodiments, heat required for the catalytic reforming is provided directly by inductive heating. Inductive heating mechanisms may include eddy currents, magnetic hysteresis, and magnetic resonance. Induction heating, which provides a non-contact heat source, can be used to heat magnetic particles or electrically conductive particles, embedded in a catalyst in the reactor, or electrical current conduction coils within or around the reactor. Alternatively, materials that act as both an inductor for hysteresis heating and a catalyst for methane reforming can be used.

In certain embodiments, heat required for the catalytic reforming is provided indirectly using a heat storage medium and/or heat transfer fluid. The heat transfer fluid is a fluid (e.g., gas or liquid) that surrounds at least part of the reformer tubes and/or circulates around the outside of at least part of the reformer tubes and that can transfer at least a portion of its heat to the reformer tubes (e.g., by conduction). The low-carbon electricity can be used to heat the heat transfer fluid directly (e.g., using a resistive or inductive heater), thereby making the use of the heat storage medium optional. Alternatively, or additionally, the low-carbon electricity can be used to heat a heat storage medium. The heat storage medium is configured to store excess thermal energy (e.g., generated by the low-carbon electricity) so that it can be used hours, days, or weeks later to heat the reformer tubes (e.g., directly or via the heat transfer liquid). In particular, the heat storage medium is configured to be able to store and provide heat at the relatively high temperatures required for methane reforming (e.g., greater than about 500° C.). The heat storage medium may be in the form of a molten salt or a particulate solid material. As will be understood by those skilled in the art, some molten salts will have characteristics, such as melting point, which make them suitable for methane reforming reactions. Molten salts may be suitable for use as both a heat transfer fluid and/or a heat storage medium. For example, the reformer can be configured such that molten salt surrounds at least a portion of the tubes. In certain embodiments, the heat for the methane reforming is provided using both a heat transfer liquid and a heat storage medium containing molten salts. In certain embodiments, the heat for the methane reforming is provided using a heat storage medium based on particulate solid material (e.g., hot sand). For example, a circulating system may convey the particles to a heater, where they are heated using low-carbon electricity and fed to high temperature particle silos, where they are stored until fed (e.g., by gravity) to a heat exchanger (e.g., pressurized fluidized bed heat exchanger), and recirculated back to the heater. The heat exchanger transfers the heat from the particles to the heat transfer fluid, which may be a gas, which in turn provides the heat to the methane reforming. Advantageously, such configurations may be used to retrofit conventional fired steam methane reformers. For example, the conventional furnace can be used to hold the heat transfer fluid around at least a portion of the reforming tubes. Using a heat storage medium is advantageous in that it provides a solution for the intermittent nature of renewable energy sources (e.g., can be used to provide heat for reforming when solar radiation is below optimum). In addition, it can provide a consistent and/or uniform heat.

In addition to methane reforming, the hydrogen production includes a hydrogen purification process. In the hydrogen purification process, the syngas produced from methane reforming (e.g., following WGS) is subjected to processing wherein hydrogen is separated from carbon monoxide, carbon dioxide, and/or methane in one or more stages to produce a stream enriched in hydrogen (i.e., containing at least 80% hydrogen). For example, in one embodiment, the hydrogen purification produces an enriched hydrogen stream having a hydrogen content of at least 90, 92, 94, 96, 98, 99, or 99.5%. In one embodiment, the hydrogen purification produces an enriched hydrogen stream having a hydrogen content of at least 99.9%. Without being limiting, some examples of suitable hydrogen purification technologies include, but are not limited to: a) absorption, b) adsorption, c) membrane separation, d) cryogenic separation, and/or e) methanation. Some examples of absorption systems that may be suitable include, but are not limited to, a monoethanolamine (MEA) unit or a methyldiethanolamine (MDEA) unit. A MEA unit may include one or more absorption columns containing an aqueous solution of MEA at about 30 wt %. The outlet liquid stream of solvent may be treated to regenerate the MEA and separate carbon dioxide. Some examples of adsorption systems that may be suitable include, but are not limited to, systems that use adsorbent bed (e.g., molecular sieves, activated carbon, active alumina, or silica gel) to remove impurities such as methane, carbon dioxide, carbon monoxide, nitrogen, and/or water from the syngas. Methanation is a catalytic process that can be conducted to convert the residual carbon monoxide and/or carbon dioxide in the syngas to methane. For example, see Eqs. 1 and 2. Since the methanation reaction consumes hydrogen, a hydrogen purification unit that includes a methanation may include carbon dioxide removal prior to methanation.

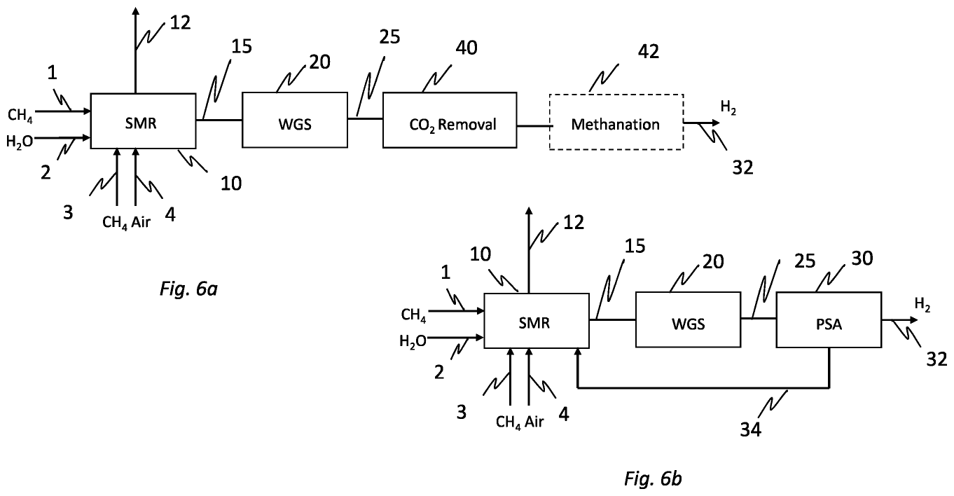

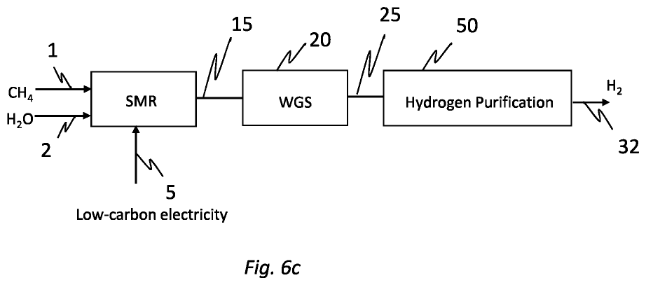

In general, the configuration of the hydrogen production process and/or plant can be dependent on the type of the methane reforming and/or hydrogen purification process provided. For example, consider the two hydrogen production processes based on SMR as illustrated in FIGS. 6a and 6b.

In FIG. 6a, the preheated feed stream 1 is fed, along with steam 2, into the reactor tubes for the SMR 10, which contain the reforming catalyst. Fuel 3 (e.g., natural gas, biomethane, refinery gas, liquid petroleum gas (LPG), and/or light naphtha, etc.) and combustion air 4 are fed into the SMR burners, which provide heat required for the endothermic reforming reaction. The syngas 15 produced from the SMR is fed to WGS 20 to produce more hydrogen. The resulting syngas 25, which may also be referred to as shifted gas, is cooled (not shown) and subjected to a hydrogen purification process. In this case, the hydrogen purification process includes a wet scrubbing carbon dioxide removal process 40 (e.g., amine absorption and regeneration cycle), and optionally includes a methanation process 42 to convert any remaining carbon monoxide and/or carbon dioxide to methane.

In FIG. 6b, the hydrogen purification process includes pressure swing adsorption (PSA) 30. The PSA 30 produces a stream enriched in hydrogen 32 and tail gas 34. The tail gas 34, which may contain unconverted methane, hydrogen, carbon dioxide, and/or carbon monoxide, is fed back to SMR 10, where it is used to provide additional process heat for the SMR (e.g., fuel the SMR burners). More specifically, the tail gas 34 is combusted together with the fuel 3. The use of PSA, and more specifically, the recycle of the tail gas to fuel the SMR burners, is generally associated with improved energy efficiency as less fuel 3 is required.

As discussed herein, the feedstock for hydrogen production includes upgraded biogas and preferably contains biomethane. It can be advantageous to use biomethane (e.g., relative to biogas that does not qualify as biomethane) because existing methane reformers may be configured to process natural gas and/or may operate more efficiently for biomethane and/or natural gas. For example, biogas that fails to qualify as biomethane may include impurities that poison the reforming catalysts. In addition, using biomethane facilitates providing the biomethane via a natural gas distribution system (e.g., the natural gas grid).

In certain embodiments, the feedstock for hydrogen production includes biomethane in addition to one or more other gases (e.g., non-renewable methane-containing gas such as fossil-based natural gas, refinery gas, liquid petroleum gas (LPG), light naphtha, etc.). Providing feedstock for hydrogen production that contains both biomethane and non-renewable methane-containing gas may provide scaling advantages for producing fuels, fuel intermediates, or products (e.g., chemical products) from biomass feedstock. When feedstock for hydrogen production includes both biomethane and non-renewable methane-containing gas, the biomethane can be allocated as either feed for the methane reforming and/or as fuel for providing heat for the methane reforming (e.g., if present). The allocation can be conducted by physically directing it to either the reforming tube(s) or the burners, or using mass balance. In certain embodiments, the biomethane is allocated disproportionally between feed for the methane reforming and/or fuel for providing heat for the reforming (e.g., all of the biomethane provided for the methane reforming or all of the biomethane provided for fuel for providing heat for the reforming). In certain embodiments, all or at least some of the biomethane is subjected to methane reforming. In certain embodiments, all or at least some of the biomethane is used as fuel for the reforming.

In certain embodiments, the biomethane is provided as feed for the methane reforming, and all or some of the heat for methane reforming is produced using low-carbon electricity.

The hydrogen production process produces hydrogen. Advantageously, the hydrogen produced using upgraded biogas (e.g., biomethane) may be considered to have environmental benefits (e.g., may be renewable hydrogen and/or low carbon hydrogen). Low carbon hydrogen, may for example, have a carbon intensity lower than about 10 gCO₂e/MJ, lower than about 5 gCO₂e/MJ, lower than about 0 gCO₂e/MJ, lower than about 0 kgCO₂e/kg H₂, lower than about 0.45 kgCO₂e/kg H₂, or lower than about 1.5 kgCO₂e/kg H₂. Hydrogen, which can be used in gas or liquid form, is very versatile as it can be used as a fuel, converted into electricity, and/or converted to one or more fuels, fuel intermediates, or chemical products. For example, hydrogen can power fuel cell electric vehicles (FCEVs), which emit no tailpipe emissions other than water, can be run through a fuel cell to power the electricity grid, or used as rocket fuel.

In certain embodiments of the disclosure, the hydrogen 161 is provided as a product 162 (e.g., for use in a fuel cell or a fuel). For example, the hydrogen can be used for transportation purposes, for generating electricity, and/or for use in district heating.

### Using the Hydrogen to Produce Fuel, Fuel Intermediate, Chemical Product

In certain embodiments, the hydrogen is provided as feedstock in a production process that produces a fuel, fuel intermediate, chemical product, or any combination thereof. A fuel refers to a material (e.g., solid, liquid, or gaseous), which may contain carbon, which can be combusted to produce power and/or heat (e.g., may be a transportation or heating fuel). A fuel intermediate is a precursor used to produce a fuel by a further conversion process, such as by a biologic conversion, a chemical conversion, or a combination thereof. A chemical product refers to a chemical compound used in a production process or a product such as a commodity. An example of a chemical product produced from hydrogen is fertilizer.

In certain embodiments, the hydrogen is provided as feedstock 163 to produce a fuel selected from long-haul trucking fuel (e.g., diesel), shipping fuel (e.g., heavy fuel oil), aviation fuel (e.g., kerosene, jet fuel) or district heating fuel. In certain embodiments, the hydrogen is provided as feedstock 163 to produce fuels or chemical products such as ammonia or fertilizer. Without being limiting some examples of suitable processing 170 are shown in FIGS. 1-4.

In certain embodiments, the hydrogen is used to produce ammonia in a Haber-Bosch process 171. In the Haber-Bosch process, which is well-known to those skilled in the art, nitrogen is converted to ammonia according to the following reaction:

N₂+3H₂→2NH₃ (8)

The reaction is conducted under high temperatures and pressures with a metal catalyst. Ammonia has an important role in the agricultural industry for production of fertilizers. Ammonia may also be used as an energy carrier for energy storage and transportation.

In certain embodiments, the hydrogen is used to produce one or more alcohols via gas fermentation 172. In gas fermentation, which is well-known to those skilled in the art, a gas mixture typically containing hydrogen with carbon dioxide and/or carbon monoxide is fed into a fermentation tank. In this embodiment, the carbon monoxide in the syngas functions as a substrate for the biologic conversion, which utilizes microorganisms or other biocatalysts. For example, acetogenic microorganisms can be used to produce a fermentation product from carbon monoxide. The production of ethanol by the acetogenic microorganisms proceeds through a series of biochemical reactions. Without being bound by any particular theory, the reactions carried out by the microorganism are as follows:

6CO+3H₂O→CH₃CH₂OH+4CO₂ (9)

6H₂+2CO₂→CH₃CH₂OH+3H₂O. (10)

Some examples of strains that can produce ethanol from syngas are those from the genus Clostridium. In addition to ethanol, Clostridium bacteria may produce significant amounts of acetic acid (or acetate, depending on the pH) in addition to ethanol, depending upon process conditions. Such conditions can be readily selected by those of skill in the art and it should be appreciated that the invention is not constrained by any particular set of parameters selected for fermentation to improve productivity.

The fermentation products produced from gas fermentation, such as methanol, ethanol, or butanol, may be used as a fuel, or may be used to produce a fuel or chemical product. For example, ethanol may be used as a fuel directly or may be blended with gasoline. In addition, some technologies are able to convert various alcohols, including ethanol, into gasoline, diesel and jet fuel blendstocks, as well as produce benzene and/or toluene. In the embodiments including gas fermentation, the hydrogen may be used to supplement another gas feed containing carbon monoxide and/or carbon dioxide.

In certain embodiments, the hydrogen is used to produce methanol 173. For example, methanol can be produced by directly hydrogenating pure carbon dioxide with hydrogen using Cu/ZnO-based catalysts. Alternatively, hydrogen can be used to produce methanol according to the following reactions:

CO₂+H₂→CO+H₂O (reverse water gas shift) (11)

CO+2H₂→CH₃OH (12)

The methanol can be used as a fuel (e.g., mixed with gasoline) or can be used to produce a fuel (e.g., biodiesel).

In certain embodiments, the hydrogen is used to produce gasoline, diesel, and/or waxes using the Fischer-Tropsch process 174. The Fischer-Tropsch process refers to a collection of chemical reactions that converts syngas into liquid hydrocarbons, typically in the presence of metal catalysts under elevated pressures and temperatures. The Fischer-Tropsch process is well known. In the embodiments including a Fischer-Tropsch process, the hydrogen may be used to supplement another gas feed containing carbon monoxide and/or carbon dioxide in order to provide the required H₂:CO (e.g., about 2).

In certain embodiments, the hydrogen is used in the hydroprocessing (e.g., hydrocracking and/or hydrotreating) 175 of renewable fats and/or oils (e.g., algae, jatropha, tallows, camelina, pyrolysis oil produced from biomass, etc.) to produce, for example, gasoline, diesel, and/or jet fuel. Such embodiments are particularly advantageous as the renewable fuels can have reduced carbon intensity and/or be fully renewable.

In certain embodiments, the hydrogen is used in the hydroprocessing (e.g., hydrocracking and/or hydrotreating) 175 of crude-oil derived liquid hydrocarbon. For example, in certain embodiments, the hydrogen (e.g., at least the renewable hydrogen) is incorporated into a crude-oil derived liquid hydrocarbon to produce, for example, gasoline, diesel, and/or jet fuel having renewable content (e.g., see U.S. Pat. Nos. 8,658,026, 8,753,854, 8,945,373, 9,040,271, 10,093,540, 10,421,663, and 10,723,621, 10,981,784). The term “crude oil derived liquid hydrocarbon”, as used herein, refers to any carbon-containing material obtained and/or derived from crude oil that is liquid at standard ambient temperature and pressure. The term “crude oil”, as used herein, refers to petroleum extracted from geological formations (e.g., in its unrefined form). Crude oil includes liquid, gaseous, and/or solid carbon-containing material from geological formations, including oil reservoirs, such as hydrocarbons found within rock formations, oil sands, or oil shale. The term “renewable content”, as used herein, refers to the portion of the fuel(s) that is recognized and/or qualifies as renewable (e.g., a biofuel) under applicable regulations. As will be understood by those skilled in the art, the quantification of the renewable content can be determined using any suitable method and is typically dependent upon the applicable regulations.

While producing a hydrogen product, such as a renewable hydrogen product, is advantageous, it is particularly advantageous when the renewable hydrogen is used as feedstock for a production process (e.g., to produce a fuel, fuel intermediate, or chemical product). It can be particularly advantageous when the renewable hydrogen is used as feedstock for producing a transportation fuel. Using the renewable hydrogen in a production process can reduce GHG emissions associated with production process, and when the production process produces a fuel, can impart renewable content to the fuel and/or reduce the carbon intensity of the fuel. The GHG reductions can be significant, particularly when the renewable hydrogen has a negative carbon intensity. Advantageously, using renewable hydrogen in the hydroprocessing of crude-oil derived liquid hydrocarbon 175 can produce long-haul trucking fuel (e.g., diesel), shipping fuel (e.g., heavy fuel oil), aviation fuel (e.g., kerosene, jet fuel) and/or district heating fuel (e.g., heating oil). Such fuels can replace and/or be used to displace the corresponding petroleum based fuel (e.g., are drop-in fuels). Further advantageously, such fuels can be produced at existing oil refineries using existing equipment. In one embodiment, the renewable hydrogen is used in the hydroprocessing (e.g., hydrocracking and/or hydrotreating) of crude-oil derived liquid hydrocarbon to produce aviation fuel having renewable content. This embodiment is particularly advantageous as it could help decarbonize commercial air travel and/or extend the life of older aircraft types by lowering their carbon footprint.

### Ccs

In general, carbon-containing material derived from the biomass can be stored using carbon capture and storage (CCS). Carbon capture and storage (CCS) is a climate change mitigation technology that leads to a reduction in atmospheric carbon dioxide relative to the option of not using the technology. In general, CCS refers to one or more processes wherein carbon dioxide is captured from the atmosphere, or captured from a process that otherwise would release it to the atmosphere, and wherein the captured carbon is stored and/or used in a way that reduces the level of carbon dioxide in the atmosphere.

One example of CCS is where carbon dioxide is captured from an emitting source and then permanently stored underground. Another example of CCS is where carbon dioxide is captured and provided as a substitute to fossil-based carbon dioxide in an application that consumes fossil-derived carbon dioxide that is extracted or produced for the primary purpose of serving such application. In such an instance, the extraction or production is avoided, and the captured carbon dioxide that would otherwise be released does not enter the atmosphere, creating a reduction in atmospheric carbon dioxide levels relative to baseline of releasing the carbon dioxide. In managing the use of carbon dioxide to applications, distribution systems (e.g., pipelines) are often used to transport the carbon dioxide. One such use is in enhanced oil recovery (EOR) projects, where high-pressure carbon dioxide is injected into wells to carry more oil to the surface. Frequently, at least some of the carbon dioxide in the distribution system is fossil-based carbon dioxide obtained from naturally occurring underground carbon dioxide deposits. Injecting a quantity of captured carbon dioxide into such carbon dioxide distribution systems can prevent an equal quantity of carbon dioxide from being removed from the naturally occurring underground deposits, and result in a reduction atmospheric carbon dioxide levels by avoiding the release of such captured carbon dioxide.

For purposes herein, the phrase “carbon capture and storage” or “CCS” refers to carbon capture with substantially permanent storage (e.g., sequestration in geological formations) and/or carbon capture and use in beneficial applications (e.g., that consume carbon dioxide or use carbon dioxide to make a product), such that there is a reduction in atmospheric carbon dioxide relative to the absence of such carbon capture and storage and/or use. For purposes herein, providing carbon-containing material (e.g., gas such as carbon dioxide, liquid such as bio-oil, or solid such as biochar) as part of carbon capture and storage refers to providing the carbon-containing material for substantially permanent storage (e.g., sequestration in geological formations) and/or use in beneficial applications (e.g., that consume carbon dioxide or use carbon dioxide to make a product), such that there is a reduction in atmospheric carbon dioxide relative to the absence of carbon capture and storage and/or use. As will be understood by those skilled in the art, it can be advantageous for the carbon capture and storage technology to be selected such that it is recognized by the applicable regulatory authority for reducing lifecycle GHG emissions and/or mitigating climate change. For example, some regulations may require storage to have a maximum leakage rate (e.g., monitoring of carbon dioxide leakage from storage for a certain time period may be mandatory).

While CCS is often discussed in terms of directly capturing carbon dioxide using one or more carbon dioxide capture technologies such as adsorption, absorption, membrane, cryogenic, and/or chemical looping technologies, in some cases the carbon dioxide is captured from the atmosphere and converted to biomass via photosynthesis and at least part of the corresponding plants are used to produce bioenergy that makes biogenic carbon available for subsequent CCS. When such bioenergy production is integrated with CCS, this may be referred to as bioenergy with carbon capture and storage or BECCS. BECCS, which is a group of technologies that combine extracting bioenergy from biomass with CCS, has the potential to provide negative GHG emissions and thus may play an important role in commitments to reach net-zero carbon emissions. For example, in some cases, BECCS can be viewed as a process where biomass (e.g., plants) is used to capture carbon dioxide from the atmosphere, the biomass is processed to produce bioenergy (e.g., heat, electricity, fuels) while releasing carbon dioxide, and the carbon dioxide produced during the processing is captured and stored such that there is there is a net transfer of carbon dioxide from the atmosphere to storage. Alternatively, or additionally, carbon-containing material derived from the biomass (i.e., other than carbon dioxide) can be stored so as to prevent or delay such carbon from being released to the atmosphere (e.g., as methane and/or carbon dioxide).

In general, carbon capture and storage (CCS) in the instant disclosure includes storing and/or using carbon-containing material at least partially derived from the biomass (e.g., containing carbon captured from the atmosphere via photosynthesis) in one or more CCS processes. In general, the carbon-containing material can be provided as gas, liquid, and/or solid carbon-containing materials. In certain embodiments, the CCS also includes storing and/or using fossil-based carbon-containing material in the one or more CCS processes. As will be understood by those skilled in the art, the carbon capture and storage technology used may be dependent on the type of carbon-containing material, the process, and/or applicable regulations (e.g., used to calculate lifecycle GHG emissions and/or qualify for fuel credits).

In certain embodiments, the CCS (e.g., 150) includes providing carbon dioxide produced from the process (e.g., produced from an ethanol fermentation process, produced from biomethane production process, produced from processing a residue of the biomethane production, and/or produced from hydrogen production) for storage and/or use as part of at least one carbon capture and storage process. In such embodiments, the carbon dioxide, which typically includes biogenic carbon dioxide (e.g., derived from the biomass), can also include fossil-derived carbon dioxide (e.g., if hydrogen production uses feed containing fossil-based methane-containing gas and biomethane). In general, the carbon dioxide can be captured using any suitable separation technology that can remove carbon dioxide from a gas mixture (e.g., biogas, syngas, flue gas). Alternatively, if the carbon dioxide is relatively pure, capturing the carbon dioxide can simply refer to collecting the carbon dioxide (e.g., in a pipe). It can be particularly advantageous to use gas separation techniques that provide a relatively pure carbon dioxide stream. Such techniques may for example, include vacuum PSA (VPSA), absorption processes (e.g., based on amines), and/or cryogenic separations (e.g., using temperatures below −10° C. or below −50° C.).

In certain embodiments, at least some of the carbon dioxide provided as part of CCS is provided for storage (e.g., sequestration) in a subsurface formation (e.g., is trapped in geological formations, such as saline aquifers, oil and natural gas reservoirs, unmineable coal seams, organic-rich shales, or basalt formations). In certain embodiments, at least some of the carbon dioxide provided as part of CCS is provided for use in enhanced oil recovery (EOR). In certain embodiments, at least some of the carbon dioxide provided as part of CCS is provided for storage in a product (e.g., mineral sequestration). For example, carbon dioxide can react with metal oxides, such as magnesium and/or calcium oxides, to produce carbonates. Such mineral carbonates have many applications. Other products may include building materials such as cement, concrete, or aggregates, chemicals, fuels, and/or food and beverages.

Capture and storage of carbon dioxide, which is well-known in the art, may include one or more gas separation processes (e.g., used to separate the carbon dioxide from one or more other components of a gas mixture and/or to produce a stream of carbon dioxide that is of sufficient purity for storage, use, and/or transport). Carbon capture and storage of carbon dioxide often include compression of the carbon dioxide and/or transport of the carbon dioxide.

Referring to FIGS. 1, 2, 3, and 4, carbon dioxide that is stored and/or used as part of CCS 150 can include carbon dioxide produced from biomethane production 120 and/or hydrogen production 160. Alternatively, or additionally, carbon dioxide produced from treating one or more byproducts of the process (e.g., digestate) can be provided as part of CCS. For example, at least part of the digestate may be subjected to one or more processes such as combustion, gasification, pyrolysis, and/or wet oxidation that produces carbon dioxide, which can be captured. In certain embodiments, a carbon capture and/or storage process may be integrated with and/or overlap another process. For example, the capture of carbon dioxide may correspond to one or more steps of the biomethane production (e.g., carbon dioxide scrubbing from methanation or biogas upgrading).

In certain embodiments, the CCS includes providing carbon dioxide produced from biomethane production for storage and/or use as part of at least one carbon capture and storage process. For example, in certain embodiments, the CCS includes providing carbon dioxide produced from anaerobic digestion as part of CCS. Such embodiments can be advantageous because some or all of the technologies used to upgrade the biogas produced by anaerobic digestion can also be used in the production of carbon dioxide suitable for storage and/or use.

In certain embodiments, the CCS includes storing carbon as a liquid and/or solid carbon-containing material derived from (i.e., obtained from or produced from) a part of the biomass not converted to bioenergy, a part of the biomass not converted to biogas and/or biomethane, and/or residue of the process (e.g., of the biomethane process). In certain embodiments, the carbon-containing material is not biodegradable under the storage conditions. In certain embodiments, the storage is selected such that if the carbon-containing material does degrade, that GHGs released from the degradation are trapped. In certain embodiments, the CCS includes storing the carbon as a solid such as biochar. Biochar, which can be produced from gasification and/or pyrolysis of the biomass, can be recycled within the gasification and/or pyrolysis processes (e.g., to provide additional fuel for the process). Alternatively, biochar, which is biologically unavailable, can be provided as a soil amendment where it can store the carbon in the soil for centuries. In certain embodiments, the CCS includes providing biochar as a soil amendment (e.g., instead of recycling it within the process), or includes subjecting a carbon-containing material derived from the biomass and not converted to bioenergy (e.g., a portion of the digestate) to gasification and/or pyrolysis, and providing the biochar produced therefrom for soil amendment or some other external use. Advantageous, such process may also produce additional bioenergy from the biomass (e.g., fuel and/or electricity). In certain embodiments, the heat and/or electricity generated from gasification and/or pyrolysis of a byproduct is used within the process (e.g., in the biomethane production process) in order to keep the carbon intensity of the biomethane, hydrogen (e.g., renewable), and/or fuel produced therefrom below a certain limit (e.g., below 20, 10 or 0 gCO₂e/MJ).

In certain embodiments, the CCS includes storing the carbon in a product. In this case, a carbon-containing material obtained and/or derived from the biomass is used to produce a product that makes the carbon unavailable for biodegradation (e.g., can be provided in products that provide continued sequestration benefits, such as building materials).

In certain embodiments, the CCS includes sequestering a liquid and/or solid carbon-containing material derived from a part of the biomass not converted to bioenergy, a part of the biomass not converted to biogas and/or biomethane, and/or residue of the process (e.g., of the biomethane process)). Such materials can be sequestered indefinitely in a subsurface formations. For example, digestate can be subjected to a hydrothermal liquefaction to provide a bio-oil that can be sequestered. The pyrolysis of biomass, which can produce biomethane, can also produce pyrolysis oil, which can be sequestered. In some cases, the sequestration method is selected to prevent biodegradation of the material and/or trap GHGs in the event of biodegradation. In some cases, the material is treated in a process to reduce the potential for biodegradation. Sequestering a liquid carbon-containing material derived from the biomass may be advantageous in that injection into the storage area may be feasible and/or there may be fewer concerns related to leakage (i.e., relative to carbon dioxide sequestration).

In certain embodiments, the CCS includes providing carbon dioxide produced from processing a part of the biomass not converted to bioenergy, a part of the biomass not converted to biogas and/or biomethane, and/or residue of the process (e.g., of the biomethane process) for storage and/or use as part of at least one carbon capture and storage process. In such embodiments, the processing can include any suitable processing, including for example, combustion, gasification, pyrolysis, and/or wet oxidation, while the residue can include any suitable material (e.g., typically liquid and/or solid) that is not converted to biogas or biomethane (e.g., digestate or biochar). In certain embodiments, the residue is waste or a byproduct of the biomethane production process. For example, in certain embodiments, the CCS includes sequestering carbon dioxide produced from processing residue, such as solid and/or liquid digestate, from the biomethane production process.

In certain embodiments, the CCS includes providing carbon dioxide produced by combusting at least a portion of the digestate for storage and/or use as part of at least one carbon capture and storage process. For example, in some embodiments, the digestate is subjected to a solids-liquid separation that provides a solids stream and a liquid stream. Such solids-liquid separation can be conducted using a screw press, centrifuge, etc. At least part of the solids stream is then combusted. Optionally, the solids are processed prior to combustion. For example, such processing can include washing, further drying (e.g., thermal drying), and/or compression (e.g., formed into bales, pellets, or brickettes). Optionally, at least a portion of the liquids stream is also combusted. For example, the liquid stream can be subjected to an evaporation process to produce relatively clean water that can be recycled back to the digester, thereby reducing water requirements while also reducing the amount of salts and/or trace metals (e.g., potassium, sodium, chromium, etc.) in the recycled water. This can increase biogas production (e.g., by removing inhibitors) and/or reduce lifecycle GHG emissions. The residue from evaporation may be provided for combustion.

Advantageously, the combustion of digestate can generate heat and/or power for the process (e.g., without requiring a substantial about of additional heat and/or power). For example, electricity can be produced by combusting at least part of the digestate in a boiler configured to produce high pressured steam for electricity generation. Optionally, at least part of digestate is combusted with another material, such as biomass from a different feedstock (e.g., wood chips). As will be understood by those skilled in the art, the combustion of digestate and/or the other material can produce a flue gas containing carbon dioxide, which can be captured and provided for storage and/or use as part of carbon capture and storage 150 to further reduce the lifecycle GHG emissions of the biomethane, renewable hydrogen produced from the biomethane, and/or fuel, fuel intermediate, or chemical produced form the biomethane and/or hydrogen. In addition, the use of heat and/or power produced by the combustion can further reduce the lifecycle GHG emissions of the biomethane, renewable hydrogen produced from the biomethane, and/or fuel, fuel intermediate, or chemical produced form the biomethane and/or hydrogen. The combustion of at least the solids component of the digestate can be advantageous because it can contain a significant amount of lignin, the energy content of which otherwise would be wasted. In addition, the combustion of digestate may be advantageous over the combustion of raw biomass, as the upstream processing may result in fewer alkali salts (e.g., potassium salts) being present during the combustion (e.g., relative to combustion of raw biomass).

In certain embodiments, the CCS includes providing carbon dioxide produced from a process that includes subjecting at least a portion of the digestate to gasification and/or pyrolysis for storage and/or use as part of at least one carbon capture and storage process. For example, in some embodiments, the digestate is subjected to a solids-liquid separation that provides a solids stream and a liquid stream. Such solids-liquid separation can be conducted using a screw press, centrifuge, etc. At least part of the solids stream is then subjected to gasification and/or pyrolysis. Optionally, the solids are processed prior to gasification and/or pyrolysis. For example, such processing can include washing, further drying (e.g., thermal drying), and/or compression (e.g., formed into bales, pellets, or brickettes). Advantageously, the gasification and/or pyrolysis of the digestate produces syngas that can be used in fuel cells to produce electricity for the process, or can be combusted to generate heat and/or power for the process. Further advantageously, the syngas contains carbon dioxide, which can be captured (e.g., pre- or post-combustion) and provided for storage and/or use as part of at least one carbon capture and storage process to further reduce the lifecycle GHG emissions of the biomethane, renewable hydrogen produced from the biomethane, and/or fuel, fuel intermediate, or chemical produced form the biomethane and/or hydrogen. Producing heat and/or power from the combustion of syngas can be advantageous over producing heat and/or power from the combustion of digestate, because such electric power can be generated in engines and/or gas turbines, which may be cheaper and more efficient that the steam cycle used in incineration, and that the carbon dioxide can be captured from the syngas (i.e., pre-combustion) rather than post-combustion. For example, electricity can be produced by combusting at least part of the syngas using Stirling-engine based combined heat and power (CHP) technology. In certain embodiments, carbon dioxide is captured pre-combustion, thereby enabling the capture of carbon dioxide from gas streams having relatively high carbon dioxide contents and/or pressures, while also providing a stream enriched in hydrogen for combustion and/or for use in one or more fuel cells. In certain embodiments, the gasification and/or pyrolysis of at least part of the digestate produces a residue (e.g., waste and/or byproduct), at least part of which is combusted, thereby producing carbon dioxide that can be provided as part of carbon capture and storage. For example, gasification and/or pyrolysis can produce biochar that can be combusted, while pyrolysis can also produce biooil that can be combusted. In certain embodiments, the digestate, or a stream derived therefrom, is processed with fossil fuels. For example, solid digestate may be gasified with coal, while pyrolysis oil may be converted to electrical power through co-combustion in a conventional fossil fuel power plant.

In certain embodiments, the CCS includes providing carbon dioxide produced from a process that includes subjecting at least a portion of the digestate to wet oxidation for storage and/or use as part of at least one carbon capture and storage process. Advantageously, such wet oxidation can produce carbon dioxide that can be captured and provided as part of carbon capture and storage to further reduce the lifecycle GHG emissions of the biomethane, renewable hydrogen produced from the biomethane, and/or fuel, fuel intermediate, or chemical produced form the biomethane and/or hydrogen.

Advantageously, providing carbon-containing material that is a residue, or is produced from processing residue of the biomethane production process, can increase the amount of biogenic carbon from the biomass associated with carbon capture and storage. The resulting reduction in GHG emissions is significant when the captured carbon is derived from the digestate. Without being limiting in any way, and depending on the feedstock and/or process, about 50% of the carbon from the original biomass may end up in the biogas (e.g., as CO₂ and CH₄) while about 50% may end up in the digestate. Accordingly, providing carbon dioxide derived from the digestate (e.g., produced from combusting the digestate) as part of carbon capture and storage can significantly decrease the lifecycle GHG emissions of the biomethane, renewable hydrogen produced from the biomethane, and/or fuel, fuel intermediate, or chemical produced form the biomethane and/or hydrogen. Further advantageously, processing a residue of the biomethane production can facilitate the use the carbon from the biomass that otherwise would not be used as and/or converted to bioenergy (e.g., heat, power, or biofuel, including, for example, biomethane, hydrogen, gasoline, diesel, jet fuel).

In certain embodiments, the CCS includes storing carbon dioxide produced from hydrogen production. In general, carbon dioxide can be captured from any suitable part of the hydrogen production process. The hydrogen production processes in FIGS. 6a and 6b both emit carbon dioxide in the flue gas 12. In the older style unit illustrated in FIG. 6a, the carbon dioxide emitted in the flue gas is produced from the combustion of the fuel 3 used to fire the SMR furnaces. In the newer style unit illustrated in FIG. 6b, the carbon dioxide emitted in the flue gas is produced from both the methane reforming and the combustion of the fuel 3 used to fire the SMR furnace. In the hydrogen production processes illustrated in FIGS. 6a and 6b, the carbon dioxide can be captured from the syngas 25 (e.g., using vacuum pressure swing adsorption (VPSA) or an absorption amine unit) and/or from the flue gas 12 (e.g., using an activated amine process). In the hydrogen production processes illustrated in FIG. 6b, the carbon dioxide can additionally or alternatively be captured from the tail gas 34. In general, it may be more technically and/or economically more feasible to capture the carbon dioxide from the syngas or tail gas, as the flue gas may have a relatively low carbon dioxide concentration (e.g., relatively low partial pressure) and/or may be at a lower pressure (e.g., atmospheric). In addition, the flue gas may contain nitrogen (e.g., CH₄/N2 separations can be more challenging than CH₄/CO₂ separations). In the hydrogen production system illustrated in FIG. 6c, the heat for SMR is provided from low-carbon electricity. Since such systems do not produce flue gas, the carbon dioxide from hydrogen production is only captured from the syngas and/or tail gas, if present.

In certain embodiments of the instant disclosure, the hydrogen production process includes methane reforming using at least one reactor configured to provide electrically generated heat for methane reforming, and carbon dioxide generated at least partially in such reactor(s) is provided for storage and/or use in at least one CCS process.

Advantageously, the combination of CCS with the hybrid electric hydrogen using low-carbon electricity production makes capturing most of the carbon dioxide from hydrogen production more economically and/or technically feasible. For example, as discussed in with reference to FIGS. 6a, 6b, and 6c, since there is no flue gas produced from hydrogen production in the hybrid electric process, the more economically and/or challenging task of capturing carbon dioxide from the flue gas is avoided (e.g., the capture of carbon dioxide can be conducted without combating the low carbon dioxide concentrations, low pressures, and/or presences of nitrogen in the flue gas).

In certain embodiments, the CCS includes storing carbon dioxide produced from hydrogen production and storing carbon produced during biomethane production. Advantageously, this facilitates storing a greater percentage of the carbon from the biomass not converted to bioenergy, to biogas and/or methane, and/or derived from residue (e.g., from biomethane production), which can significantly reduce GHG emissions.

Although the newer style hydrogen plants that include PSA are generally considered more efficient, in certain embodiments, it can be advantageous to use hydrogen purification that does not produce a tail gas having sufficient heat content for combustion (e.g., to not use PSA) in the hybrid-electric embodiment using low-carbon electricity. In certain embodiments, the hybrid-electric hydrogen production using low-carbon electricity includes hydrogen purification based on absorption processes and/or cryogenic process, where the carbon dioxide output may be relatively pure (e.g., for CCS) and/or where no methane and/or hydrogen rich off-gas is produced. In certain embodiments, the hybrid-electric hydrogen production using low-carbon electricity includes hydrogen purification based on adsorption, and a methane and/or hydrogen containing off-gas is used elsewhere within the process. In certain embodiments, the hybrid-electric hydrogen production using low-carbon electricity includes hydrogen purification based on adsorption, and a methane and/or hydrogen containing off-gas is at least partially recycled back as feed for the methane reforming reaction(s).

The process(es) and/or system(s) of the instant disclosure produce at least one fuel (e.g., hydrogen, biomethane, ammonia, etc.), at least one fuel intermediate (e.g., hydrogen, biomethane, ammonia, etc.), and/or at least one product (e.g., chemical product, ammonia, fertilizer, etc.). The inclusion of CCS within various embodiments of the disclosure can reduce GHG emissions from the process (i.e., relative to no CCS) and/or reduce lifecycle GHG emissions of the product(s) of the process (i.e., relative to no CCS). In certain embodiments, combining CCS with fuel production provides a fuel that has a reduced carbon intensity (i.e., relative to with no CCS). The term “carbon intensity” or “CI” refers to the quantity of lifecycle GHG emissions, per unit of fuel energy, and is often expressed in grams of CO₂ equivalent emissions per unit of fuel (e.g., gCO₂e/MJ or gCO₂e/MMBTU). As will be understood by those skilled in the art, lifecycle GHG emissions and/or carbon intensity are typically determined using Lifecycle Analysis (LCA), which identifies and estimates all GHG emissions in producing a fuel or product, from the growing or extraction of raw materials, to the production of the fuel or product, through to the end use (e.g., well-to-wheel). The LCA can reflect emissions credits and debits accrued across the whole production pathway or supply chain, including emissions effects of biomass carbon not converted into useful products. Those skilled in the art will understand that lifecycle GHG emissions and/or carbon intensity values for a given fuel or product can be dependent upon the methodology used (e.g., as required by the applicable regulatory authority).

In general, any methodology can be used to determine carbon intensity and/or lifecycle GHG emissions. However, when the fuel or product is specially treated for meeting a certain lifecycle GHG reduction threshold under certain regulations (e.g., is treated as clean or low carbon intensity hydrogen) and/or when the method includes obtaining one or more credits for the fuel or product and/or its production, the methodology will be selected to comply with the prevailing rules and regulations in the applicable jurisdiction (e.g., relevant to desired credits).

Methodologies for calculating carbon intensities and/or lifecycle GHG emissions according to various regulatory bodies are well known in the art and can be readily calculated by those of ordinary skill in the art. For example, in certain embodiments, the carbon intensities and/or lifecycle GHG emissions are determined using a LCA model, such as the GREET model. The GREET model, which is well-known by those skilled in the art, refers to “The Greenhouse gases, Regulated Emissions, and Energy use in Technologies Model” developed at Argonne National Laboratory (ANL) (e.g., greet.es.anl.gov). In certain embodiments, the carbon intensities and/or lifecycle GHG emissions are determined based on the fuel/product being produced according to a certain pathway (e.g., a fuel pathway). For example, in certain embodiments, the carbon intensities are pathway certified carbon intensities or are regulatory default value carbon intensities. In general, the term “fuel pathway” refers to a collective set of processes, operations, parameters, conditions, locations, and technologies throughout all stages that the applicable agency considers appropriate to account for in the system boundary of a complete analysis of that fuel's lifecycle greenhouse gas emissions. In some cases, a fuel pathway can be a specific combination of three components, namely: (1) feedstock, (2) production process, and (3) product or fuel type. In certain embodiments, the carbon intensities are regulatory default value carbon intensities. For example, in the UK, biomethane produced from wet manure may have a default carbon intensity of 22 gCO2 eq/MJ when the digestate is fed to an open enclosure, and when the off-gas from biogas upgrading is not combusted, or may have a default carbon intensity of −100 gCO2 eq/MJ when the digestate is fed to closed enclosure, and when the off-gas from biogas upgrading is combusted. In certain embodiments, the carbon intensities (e.g., of biomethane feedstock) are determined using disaggregated default values (e.g., associated with certain feedstocks and/or steps in a supply chain) or a mixture of disaggregated default values and measured values (e.g., based on supply chain specific measured values). In certain embodiments, the carbon intensities (e.g., of biomethane feedstock) are determined (e.g., using a LCA) and then verified by the regulatory agency (e.g., the fuel pathway and/or corresponding carbon intensities can be approved by the regulatory agency) and/or by a verification body approved and/or appointed by the regulatory agency. The carbon intensity values recited herein are determined using the CA-GREET model (e.g., see, https://ww2.arb.ca.gov/resources/documents/lcfs-life-cycle-analysis-models-and-documentation), unless otherwise specified.

In certain embodiments of the disclosure, CCS 150 includes providing carbon-containing material (e.g., carbon dioxide) obtained and/or produced from more than one point in the process (e.g., multiple CCS processes). For example, such multi-tiered CCS can include providing carbon dioxide produced from multiple biogas plants for CCS, wherein the biogas produced from such plants is used to provide the biomethane for hydrogen production, and/or can include various combinations of (a) storing carbon dioxide captured from biomethane production, (b) storing carbon dioxide captured from hydrogen production, and (c) storing gaseous, liquid, and/or solid carbon-containing material derived from a part of the biomass not converted to biogas/biomethane (e.g., from the residue of biomethane production). Using carbon capture and storage, where the carbon is captured from multiple points in the process, decreases the amount of GHG emissions attributable to producing bioenergy from the biomass. In general, such carbon capture and storage can be achieved using one or more carbon capture and storage processes.

In certain embodiments of the disclosure, the CCS 150 includes at least two CCS processes, including CCS of carbon dioxide from biomethane production (e.g., from biogas upgrading) and CCS of carbon dioxide from hydrogen production (e.g., captured from the syngas). In certain embodiments of the disclosure, the CCS 150 includes at least three CCS processes, including CCS of carbon dioxide from biomethane production (e.g., from biogas from a purification process in gasification/methanation), CCS of carbon dioxide from hydrogen production (e.g., captured from the syngas), and CCS of a byproduct of biomethane production (e.g., biochar, a carbon-containing material derived from digestate, or a combination thereof). Advantageously, this three-tiered approach can significantly reduce the lifecycle GHG emissions of the fuel (e.g., hydrogen), fuel intermediate, or chemical product produced, even without including the hybrid-electric process using low-carbon electricity. In certain embodiments of the disclosure, only CCS of carbon from biomethane production is provided (e.g., carbon dioxide from biogas and/or carbon from the digestate).

Advantageously, the hydrogen produced from biomass according to certain embodiments can have lifecycle GHG emissions that are similar to and/or lower than green hydrogen (e.g., can be about net-zero). In certain embodiments, the hydrogen production process can reduce GHG emissions (e.g., can be net negative). In certain embodiments, the carbon intensity of the fuel (e.g., renewable hydrogen or fuel produced using the renewable hydrogen) is negative and/or relatively low. In certain embodiments, the type of biomass and/or quantity of carbon to be stored is selected such that the carbon intensity of the fuel is below a predetermined value (e.g., required for regulatory purposes). For example, in certain embodiments, the type of biomass is selected to keep the carbon intensity of the hydrogen below that of green hydrogen (e.g., below zero).

In certain embodiments, the renewable hydrogen produced from the hybrid-electric process using low-carbon electricity and biomethane has a carbon intensity not more than 35 gCO₂e/MJ, 33 gCO₂e/MJ, 30 gCO₂e/MJ, or 25 gCO₂e/MJ, without accounting for CCS. In certain embodiments, the renewable hydrogen produced from the hybrid-electric process using low-carbon electricity and biomethane has a carbon intensity less than 0 gCO₂e/MJ, less than −10 gCO₂e/MJ, or less than −20 gCO₂e/MJ, when accounting for CCS. In certain embodiments of the disclosure, the CCS 150, in combination with the hybrid-electric process using low-carbon electricity and biomethane, produces renewable hydrogen having a carbon intensity that is not more than −10 gCO₂e/MJ, −20 gCO₂e/MJ, −30 gCO₂e/MJ, −40 gCO₂e/MJ, or −50 gCO₂e/MJ, of H₂. The carbon intensity values of hydrogen provided herein are calculated using the lower heating value (LHV), unless otherwise specified.

In certain embodiments of the instant disclosure, the production of hydrogen in the hybrid-electric process achieves a percentage reduction in lifecycle greenhouse gas emissions compared to a baseline hydrogen production that is at least 40%, where the baseline hydrogen production refers to hydrogen production by steam methane reforming where high-temperature steam is used to produce hydrogen from fossil-based natural gas and where there is no carbon capture and storage. In certain embodiments of the instant disclosure, the production of hydrogen in the hybrid-electric process achieves a percentage reduction in lifecycle greenhouse gas emissions compared to the baseline hydrogen production that is at least at least 75%, at least 85%, or at least 95%.

While providing zero carbon hydrogen is generally advantageous, it may be particularly advantageous if the carbon intensity is as low as possible when the hydrogen is used as a fuel or to produce a fuel, for fuel credit purposes.

In certain embodiments of the disclosure, the process includes generating, obtaining, or providing credits (e.g., fuel credits). Credits are used to incentivize renewable fuels, often in the transportation sector. For example, credits such as fuel credits can be used to demonstrate compliance with some government initiative, standard, and/or program, where the goal is to reduce GHG emissions (e.g., reduce carbon intensity in transportation fuels as compared to some baseline level related to conventional petroleum fuels) and/or produce a certain amount of biofuel (e.g., produce a mandated volume or a certain percentage of biofuels). The target GHG reductions and/or target biofuel amounts may be set per year or for a given target date. Some non-limiting examples of such initiatives, standards, and/or programs include the Renewable Fuel Standard Program (RFS2) in the United States, the Renewable Energy Directive (RED II) in Europe, the Fuel Quality Directive in Europe, the Renewable Transport Fuel Obligation (RTFO) in the United Kingdom, and/or the Low Carbon Fuel Standards (LCFS) in California, Oregon, or British Columbia). Credits can also be used to incentivize other products associated with reduced carbon or greenhouse gas emissions, such as for example, producer or production credits for clean hydrogen or credits for products made using clean hydrogen.

The term “credit”, as used herein, refers to any rights or benefits relating to GHG or carbon reduction including but not limited to rights to, credits, revenues, offsets, GHG gas rights, tax benefits, government payments or similar rights related to or arising from emission reduction, trading, or any quantifiable benefits (including recognition, award or allocation of credits, allowances, permits or other tangible rights), whether created from or through a governmental authority, a private contract, or otherwise. A credit can be a certificate, record, serial number or guarantee, in any form, including electronic, which evidences production of a quantity of hydrogen or fuel meeting certain life cycle GHG emission reductions relative to a baseline (e.g., a gasoline baseline) set by a government authority. Credits for low CI hydrogen may be set by regulatory authority and provided in many forms, e.g., producer credits and the like. Non-limiting examples of fuel credits include RINs and LCFS credits. A Renewable Identification Number (or RIN), which is a certificate that acts as a tradable currency for managing compliance under the RFS2, may be generated for each gallon of biofuel (e.g., ethanol, biodiesel, etc.) produced. A Low Carbon Fuel Standard (LCFS) credit, which is a certificate which acts as a tradable currency for managing compliance under California's LCFS, may be generated for each metric ton (MT) of CO₂ reduced.

In general, the requirements for obtaining, generating, or causing the generation of credits can vary by country, the agency, and or the prevailing regulations in/under which the credit is generated. In many cases, credit generation may be dependent upon a compliance pathway (e.g., predetermined or applied for) and/or the biofuel meeting a predetermined GHG emission threshold. For example, with regard to the former, the RFS2 categorizes biofuel as cellulosic biofuel, advanced biofuel, renewable biofuel, and biomass-based diesel. With regard to the latter, to be a renewable biofuel under the RFS2, corn ethanol should have lifecycle GHG emissions at least 20% lower than an energy-equivalent quantity of gasoline (e.g., 20% lower than the 2005 EPA average gasoline baseline of 93.08 gCO₂e/MJ). In low carbon-related fuel standards, biofuels may be credited according to the carbon reductions of their pathway. For example, under California's LCFS, each biofuel is given a carbon intensity score indicating their GHG emissions as grams of CO₂ equivalent per megajoule (MJ) of fuel, and fuel credits are generated based on a comparison of their emissions reductions to a target or standard that may decrease each year (e.g., in 2019, ethanol was compared to the gasoline average carbon intensity of 93.23 gCO₂e/MJ), where lower carbon intensities generate proportionally more credits.

In certain embodiments, the process includes monitoring inputs and/or outputs from each of the biogas production, biomethane production, hydrogen production, and/or CCS. In this case, each of the inputs is a material input or energy input and each of the outputs is a material output or an energy output. Monitoring inputs and/or outputs of these process may facilitate calculating and/or verifying GHG emissions of the process, calculating and/or verifying carbon intensity of the fuel, fuel intermediate, or chemical product, may facilitate fuel credit generation (e.g., based on volumes of fuel produced), and/or may facilitate determining renewable content (e.g., when co-processing renewable and non-renewable fuels). Monitoring can be conducted over any time period (e.g., monthly statements, etc.). Monitoring can be conducted in conjunction with and/or using any suitable technology or combination of technologies that enables measurement of material and/or energy flows.

As described herein, certain embodiments of the instant disclosure relate to hydrogen production having a relatively high efficiency and/or hydrogen with a relatively low carbon intensity (e.g., relative to green hydrogen and/or renewable hydrogen produced by the SMR of biomethane without using an electric-hybrid process using low-carbon electricity and biomethane). For example, although producing biomethane (as opposed to raw biogas or cleaned biogas) adds an additional processing steps and/or cost, it may improve the process efficiency and/or carbon intensity while exploiting infrastructure used for transporting and/or processing natural gas. In addition, it may aid in monitoring inputs and/or outputs of some of the processes. Transporting the biomethane using a natural gas distribution system may also facilitate the use of biomethane having a relative low carbon intensity. For example, while biomethane from landfill gas may have a carbon intensity of about 40-50 gCO₂e/MJ, biomethane produced from manure is typically lower (e.g., dairy manure may have CI of about −270 gCO₂e/MJ, while swine manure may have a CI that is about −350 gCO₂e/MJ). Using biomethane having a carbon intensity that is less than 0 gCO₂e/MJ can significantly reduce the carbon dioxide of hydrogen produced therefrom. In certain embodiments, the biomethane is produced from manure livestock. In certain embodiments, the biomethane has a carbon intensity less than 0 gCO₂e/MJ, less than −10 gCO₂e/MJ, or less than −20 gCO₂e/MJ of CH₄. In addition, the carbon intensity of the hydrogen and/or fuel, fuel intermediate, or chemical product produced therefrom may have a reduced carbon intensity as a result of one or more CCS processes (i.e., relative to if there is no CCS). Advantageously, the use of the hybrid-electric process using low-carbon electricity and biomethane can be combined with CCS from biogas production, biomethane production, and/or hydrogen production, thereby ensuring that a large part of the carbon from the biomass can be either captured and stored or converted to bioenergy. Since such a large part of the carbon from biomass can be stored, the hybrid-electric process using low-carbon electricity and biomethane can produce hydrogen with a negative carbon intensity, and thus can reduce GHG emissions more than the production of an equal quantity of green hydrogen. Moreover, it can do so using less renewable electricity.

For the hybrid-electric hydrogen production based on SMR of biomethane, the potential amount of carbon dioxide that can be removed from the atmosphere was calculated to be about 110 g CO₂/MJ of H₂ if the carbon dioxide from the syngas from the hydrogen production and the carbon from the biomethane production is stored. In certain embodiments, the number of storage processes in the CCS and/or the type of biomass used to produce the biomethane is selected to remove at least about 20 g CO₂/MJ of H₂, at least about 30 g CO₂/MJ of H₂, at least about 40 g CO₂/MJ of H₂, at least about 50 g CO₂/MJ of H₂, at least about 60 g CO₂/MJ of H₂, at least about 70 g CO₂/MJ of H₂, at least about 80 g CO₂/MJ of H₂, at least about 90 g CO₂/MJ of H₂, or at least about 100 g CO₂/MJ of H₂ using the hybrid-electric hydrogen production with low-carbon electricity. Advantageously, certain embodiments can provide such levels of net GHG reduction while also providing good energy efficiency from the hydrogen production (e.g., using hybrid-electric hydrogen production with biomethane). Rough calculations estimate that an energy efficiency of about 90% may be achieved.

Advantageously, the production of hydrogen in the hybrid-electric process using low-carbon electricity may achieve a percentage reduction in lifecycle greenhouse gas emissions compared to a baseline hydrogen production that is at least 40%, where the baseline hydrogen production refers to hydrogen production by steam methane reforming where high-temperature steam is used to produce hydrogen from fossil-based natural gas and where there is no carbon capture and storage. In addition, it may achieve a reduction in lifecycle greenhouse gas emissions compared to green hydrogen production. Further advantageously, the production of hydrogen in the hybrid-electric process using low-carbon electricity may reduce efficiency losses compared to green hydrogen production.

In certain embodiments of the instant disclosure, the energy products from the process (e.g., renewable hydrogen and/or process heat) are produced with greater than 82.5% energy efficiency greater than 85% energy efficiency, greater than 87.5% energy efficiency, greater than 90% energy efficiency, greater than 92% energy efficiency, greater than 94%, energy efficiency, or greater than 96% energy efficiency. Such levels of efficiency are much higher than for green hydrogen production.

Since the production of hydrogen in the hybrid-electric process using low-carbon electricity may be produced by existing and/or modified hydrogen plants, it has the potential to serve existing hydrogen markets.

The following Examples are provided to illustrate certain advantages of certain embodiments of the instant disclosure and are not intended to limit the scope of the invention(s) (e.g., the examples are provided to illustrate one or more benefits of certain embodiments of the instant disclosure related to energy efficiency, reduction in carbon emissions, and/or economics relative to conventional SMR). For purposes herein, conventional SMR refers to steam methane reforming where high-temperature steam is used to produce hydrogen from fossil-based natural gas in a fired steam methane reformer.

### EXAMPLES

### Base Case 1

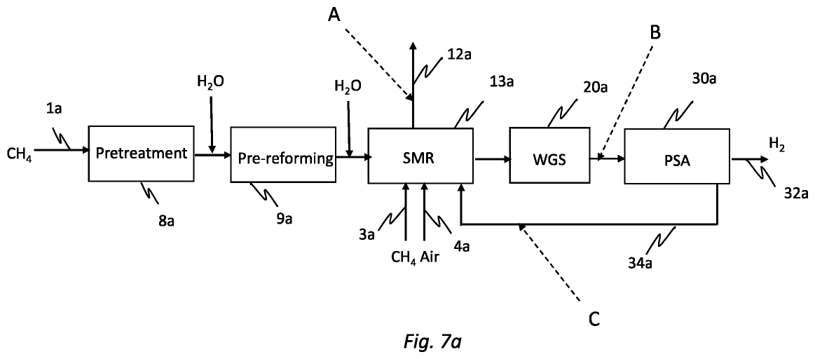

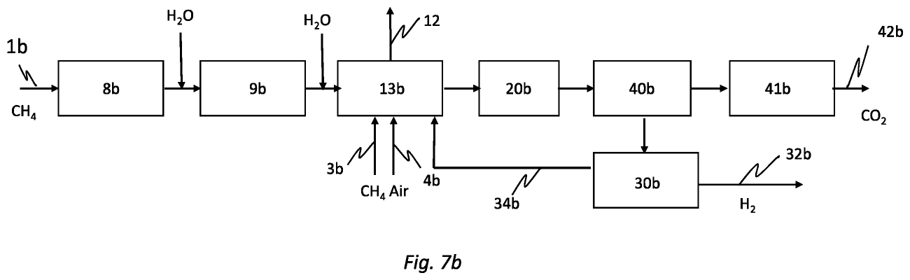

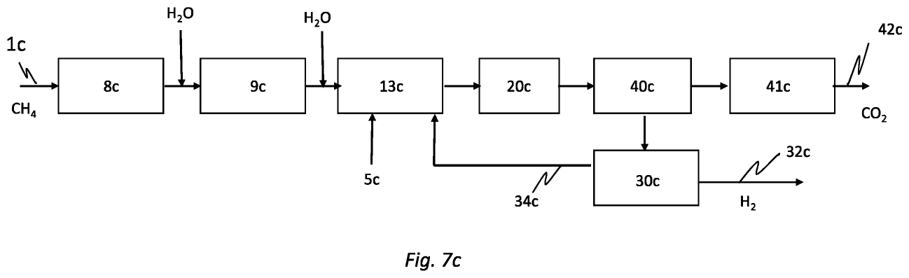

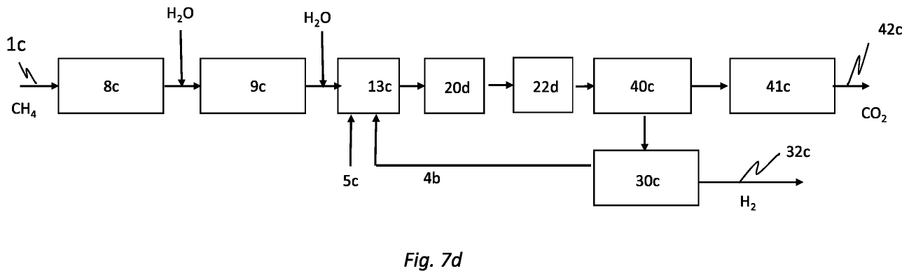

FIG. 7a shows a process for producing hydrogen according to base case 1 (i.e., conventional SMR without CCS). Natural gas 1a is pretreated 8a (e.g., to remove sulfur and/or chlorine, thereby preventing poisoning of catalysts), is subjected to pre-reforming 9a (e.g., in an adiabatic reactor to convert any heavy hydrocarbons to methane and other coproducts), and is fed to steam methane reformer 13a to produce syngas containing carbon dioxide, carbon monoxide, hydrogen, and residual methane. The resulting syngas is fed to a high temperature water gas shift reactor 20a that converts carbon monoxide to hydrogen to produce a shifted syngas. The shifted syngas is fed to PSA 30a that produces a stream enriched in hydrogen 32a. The tail gas 34a, containing a mixture of primarily carbon monoxide, carbon dioxide, methane, hydrogen, and water vapor, is fed to the burners used in the SMR 13a along with natural gas fuel 3a. Waste heat from the flue gas 12a and from the syngas between several unit operations in the process (e.g., between the SMR and WGS and between the WGS and PSA) is used to generate high pressure steam and/or for preheating the feed streams for pre-reforming 9a and/or the SMR 13a.

In this base case, which is based on an IEAGHG Technical Report titled “Techno-Economic Evaluation of SMR Based Standalone (Merchant) Hydrogen Plant with CCS”, 2017/02, February 2017, the hydrogen production process produces hydrogen at a rate of 100,000 Nm³/h (˜8,915 kg hydrogen/hr) using about 30,563 kg natural gas/hr and emits about 0.81 kg of CO₂ per Nm³ of hydrogen (e.g., emitted in the flue gas). About two thirds of the heat for reforming is obtained from the tail gas (e.g., of the 30,563 kg natural gas/hr, about 26,231 kg/hr is used for feedstock 1a, while 4,332 kg/hr is used as fuel 3a).

The hydrogen yield, kg_{H2}/kg_{NG} is given as:

$$\frac{8 , 915 kg hydrogen per hr}{30 , 563 kg natural gas per hr} = 0.29$$

The energy efficiency is given as:

$$\frac{Energy out}{Energy in} = \frac{142.9 \frac{MJ}{kg} * 8 , 915 kg}{51.6 \frac{MJ}{kg} * 30 , 563 kg + net power used} = 81. \%$$

This energy efficiency assumes that the higher heating value (HHV) of natural gas is 51.6 MJ/kg, the HHV of hydrogen is 142.9 MJ/kg, and that there is no net power used. If excess steam produced from the process is used to produce a surplus of ˜9.9 MWe electricity, which if taken into account as a negative net power use, the efficiency would be 83.5%.

### Base Case 2

Base case 2 differs from base case 1 in that it includes CCS. The capture of carbon dioxide from hydrogen production, and the technology for the same, is well known in the art. In the configuration illustrated in FIG. 7a, carbon dioxide may be captured from the flue gas (A), the shifted syngas (B), or the tail gas (C). While option (A) of capturing carbon dioxide from the flue gas has the potential to capture about 90% of the carbon dioxide produced, option (B) of capturing carbon dioxide from the shifted syngas has the potential to capture only about 56% of the carbon dioxide produced. However, option (B) can be less than half the cost of option (A), and is more common for capturing carbon dioxide from a SMR based hydrogen plant. In base case 2, the carbon is captured from the shifted syngas (e.g., option B) using MDEA-based absorption.

FIG. 7b shows a process for producing hydrogen according to base case 2 (i.e., conventional SMR with CCS). Natural gas 1b is pretreated 8b (e.g., to remove sulfur and/or chlorine, thereby preventing poisoning of catalysts), is subjected to pre-reforming 9b (e.g., in an adiabatic reactor to convert any heavy hydrocarbons to methane and other coproducts), and is fed to steam methane reforming 13b to produce syngas containing carbon dioxide, carbon monoxide, hydrogen, and residual methane. This syngas is fed to a high temperature shift reactor 20b that converts carbon monoxide to hydrogen to produce a shifted syngas. The shifted syngas is fed to MDEA-based absorption 40b, which absorbs carbon dioxide, and the resulting carbon dioxide-depleted stream is fed to PSA 30b, which produces the stream enriched in hydrogen 32b and the tail gas 34b. The tail gas 34b from the PSA, containing a mixture of primarily carbon monoxide, methane, hydrogen, and water vapor, is fed along with natural gas fuel 3b to the burners used in the SMR 13b. The carbon dioxide 42b is subjected to compression and dehydration 41b (e.g., prior to being injected into a carbon dioxide pipeline). Waste heat from the flue gas 12b and from the syngas gas (e.g., after the SMR and after the WGS) is used to produce high-pressure steam and for preheating the feed streams for pre-reforming 9b and the SMR 13b.

In base case 2, which is also based on the IEAGHG Technical Report, the hydrogen production process also produces hydrogen at a rate of 100,000 Nm³/h (˜8,915 kg hydrogen/hr) using about 31,562 kg natural gas/hr, and emits about 0.37 kg of CO₂ per Nm³ of hydrogen. The increased natural gas consumption is at least partially due to natural gas used to produce steam for regenerating the solvent used at the MDEA plant.

The use of carbon capture depresses the hydrogen yield, kg_{H2}/kg_{NG} which is given as:

$$\frac{8 , 915 kg hydrogen per hr}{31 , 562 kg natural gas per hr} = 0.28$$

The energy efficiency is given as:

$$\frac{Energy out}{Energy in} = \frac{142.9 \frac{MJ}{kg} * 8 , 915 kg}{51.6 \frac{MJ}{kg} * 31 , 562 kg + net power used} = 78. \%$$

This energy efficiency assumes that the higher heating value (HHV) of natural gas is 51.6 MJ/kg, the HHV of hydrogen is 142.9 MJ/kg, and that there is no net power used. If excess steam produced from the process were to be used to produce a surplus of ˜1.492 MWe electricity, and this was taken into account as a negative net power consumption, the efficiency would be 79.4%.

In this base case, the MDEA plant can capture about 0.47 kg of CO2 per Nm3 of hydrogen (e.g., about 56% of the CO2 produced). The carbon dioxide emissions relative to base case 1 is 46%.

### Examples 1-5

Examples 1-5 correspond to an analysis conducted to establish the effect of using electrically heated SMR and/or various other modifications relative to base case 1 and/or base case 2. The calculations assume that the hydrogen plant is a standalone facility without integration with other processes, that there is constant hydrogen output, and that analogous process steps/units are conducted similarly (e.g., the Examples use the same pretreatment/pre-reforming operations as the base cases, use the same reforming temperatures as the base cases, achieve the same PSA hydrogen recovery (90%) as the base cases, and achieve about the same carbon dioxide removal (98%) from the syngas from the base cases). For comparison purposes, it is also assumed that the hydrogen production process produces hydrogen at a rate of 100,000 Nm³/h (˜8,915 kg hydrogen/hr), has a hydrogen purity>99.9%, and is provided at 2.5 MPa and 40° C. (i.e., the same as the base cases). In embodiments that include low temperature shift (LTS), the inlet temperature for the low temperature shift (assuming a conventional Cu—ZnO LTS catalyst) is assumed to be 190° C.

### Example 1

FIG. 7c shows a process for producing hydrogen according to Example 1 (i.e., hybrid-electric SMR with CCS). Natural gas 1c is pretreated 8c (e.g., to remove sulfur and/or chlorine, thereby preventing poisoning of catalysts), is subjected to pre-reforming 9c (e.g., in an adiabatic reactor to convert any heavy hydrocarbons to methane and other coproducts), and is fed to steam methane reformer 13c to produce syngas containing carbon dioxide, carbon monoxide, hydrogen, and residual methane. In this case, the steam methane reforming 13c is at least partially heated by low-carbon electricity 5c. The syngas is fed to a high temperature shift reactor 20c that converts carbon monoxide to hydrogen to produce a shifted syngas. The shifted syngas is fed to MIDEA-based absorption 40c, which absorbs carbon dioxide, and the resulting carbon dioxide-depleted stream is fed to PSA 30c, which produces the stream enriched in hydrogen 32c and the tail gas 34c. The carbon dioxide 42c is subjected to compression and dehydration 41c (e.g., prior to being injected into a carbon dioxide pipeline). High pressure steam generated from heat from the shifted gas produced by WGS is used for pre-reforming 9b and the SMR 13b.

Example 1 differs from base case 2 in that the heat for methane reforming is provided by electricity and from combustion of at least part of the tail gas. More specifically, Example 1 assumes that there is no fuel gas (e.g., 3a/3b) combusted for the SMR, that there is no cogen plant, and that electric heat is added to support the heating loads for the SMR reactions (i.e., in addition to combusting the tail gas). The hydrogen production process produces hydrogen at a rate of 100,000 Nm³/h (˜8,915 kg hydrogen/hr) using 26,188 kg natural gas/hr, and produces about 0.70 kg of CO₂ per Nm³ of hydrogen from the SMR. In this case, the value of 26,188 kg natural gas/hr corresponds to feedstock alone. Steam for the MDEA plant is produced using a portion of the tail gas.

Since no natural gas is used as fuel for the SMR, the hydrogen yield increases (i.e., relative to base case 2). More specifically, the hydrogen yield, kg_{H2}/kg_{NG}, is given as:

$$\frac{8 , 915 kg hydrogen per hr}{26 , 188 kg natural gas per hr} = 0.34$$

The energy efficiency is given as:

$$\frac{Energy out}{Energy in} = \frac{142.9 \frac{MJ}{kg} * 8 , 915 kg}{51.6 \frac{MJ}{kg} * 26 , 188 kg + net power used} = 86 \%$$

This energy efficiency assumes that the higher heating value (HHV) of natural gas is 51.6 MJ/kg, the HHV of hydrogen is 142.9 MJ/kg, and the net power used to produce the heat for reforming (i.e., from low-carbon electricity) is about 124,740 MJ/hr (or 34.65 MW). The net power used to produce the heat for reforming is calculated from the difference between the total process heat demand (i.e., all heat duties for preheating all streams, steam generation, and heat of reaction in the reformer) and total process heat production (i.e., all heat available from cooling process streams).

In this example, the MDEA plant captures about 0.47 kg of CO₂ per Nm³ of hydrogen (e.g., about 66% of the about 0.70 kg of CO₂ produced per Nm³ of hydrogen). The carbon dioxide emissions relative to base case 1 are 28%.

### Example 2

Example 2 differs from Example 1 in that 75% of the tail gas 34c by volume is recycled to the pre-reformer 9c. The remaining 25% of the tail gas is combusted to produce steam and/or heat. The tail gas has hydrogen content of about 48%, a carbon monoxide content of about 30%, a methane content of about 16%, and relatively small amounts of carbon dioxide and nitrogen (i.e., calculated in the absence of recycling).

As a result of recycling the tail gas back into the process the hydrogen yield increases to 0.40 (e.g., relative to 0.34 in Example 1), the energy efficiency decreases to 83% (e.g., relative to 86% in Example 1), and the CCS yield increases to 91% (e.g., relative to 66% in Example 1). The carbon dioxide emissions relative to base case 1 are 7%.

In this Example, the feedstock input is adjusted to account for the recycle of the tailgas. The adjustments were calculated based on equilibrium conversions for the SMR and WGS reactions (e.g., feedstock corresponds to 22,443 kg natural gas per hour). The net power increased to 106 MW (e.g., the recycle leaves less tailgas available for combustion to provide heat, which is off-set by additional electric power). At 75% recycle, nitrogen build-up remains low (<5%) and the total mass flow changes about 10%.

### Example 3

FIG. 7d shows a process for producing hydrogen according to example 3. Natural gas 1c is pretreated 8c (e.g., to remove sulfur and/or chlorine, thereby preventing poisoning of catalysts), is subjected to pre-reforming 9c (e.g., in an adiabatic reactor to convert any heavy hydrocarbons to methane and other coproducts), and is fed to a steam methane reforming 13c to produce syngas containing carbon dioxide, carbon monoxide, hydrogen, and residual methane. In this case, the steam methane reformer 13c is heated at least partially by low-carbon electricity 5c. The syngas is fed to high temperature shift 20d followed by low temperature shift 22d to produce a shifted syngas. The shifted syngas is fed to MDEA-based absorption 40c, which absorbs carbon dioxide, and the resulting carbon dioxide-depleted stream is fed to PSA 30c, which produces the stream enriched in hydrogen 32c and the tail gas 34c. The carbon dioxide 42c is subjected to compression and dehydration 41c (e.g., prior to being injected into a carbon dioxide pipeline). High pressure steam generated from heat from the shifted gas produced by WGS is used for pre-reforming 9c and the SMR 13c.

Example 3 differs from Example 1 in that the water gas shift includes a low temperature shift 22d that follows the high temperature shift 20d. In Example 1, the high temperature shift produces a shifted gas containing a residual carbon monoxide content of around 2.5-3% volume. The low temperature shift reduces the concentration of carbon monoxide to about 0.5%, while increasing the amount of carbon dioxide and hydrogen produced. In addition, additional catalyst in the SMR causes the methane slip to be reduced to 1.5% (e.g., relative to 2.4% in Example 1), the hydrogen yield increases to 0.38 (e.g., relative to 0.34 in Example 1), the energy efficiency increases to 95% (e.g., relative to 86% in Example 1), and the CCS yield increases to 87% (e.g., relative to 66% in Example 1). The carbon dioxide emissions relative to base case 1 are 10%.

### Example 4

Example 4 differs from Example 3 in that 50% of the tail gas 34c by volume is recycled to the pre-reformer 9c (not shown). The remaining 50% of the tail gas is combusted to produce steam (e.g., for preheating). As a result of recycling the tail gas back into the process the hydrogen yield increases to 0.41 (e.g., relative to 0.38 in Example 3), the energy efficiency is 91% (e.g., relative to 94% in Example 3), and the CCS yield increases to 93% (e.g., relative to 87% in Example 3). The carbon dioxide emissions relative to base case 1 are 5%.

### Example 5

Example 5 differs from Example 3 in that 75% of the tail gas 34c by volume is recycled to the pre-reformer 9c (not shown). The remaining 25% of the tail gas is combusted to produce steam (e.g., for preheating). As a result of recycling the tail gas back into the process the hydrogen yield increases to 0.44 (e.g., relative to 0.38 in Example 3), the energy efficiency is 89% (e.g., relative to 94% in Example 3), and the CCS yield increases to 96% (e.g., relative to 87% in Example 3). The carbon dioxide emissions relative to base case 1 are 2%.

The impacts of using electric heating, tail gas recycling, and/or low temperature shift on hydrogen yield, energy efficiency, and/or CCS yield are summarized in Table 1.

TABLE 1

| | Base | Base | Example | Example | Example | Example | Example |
|---|---|---|---|---|---|---|---|
| | case 1 | case 2 | 1 | 2 | 3 | 4 | 5 |
| SMR heat | NG + | NG + | Electric | Electric | Electric | Electric | Electric |
| source | tail gas | tail gas | power + | power + | power + | power + | power + |
| | | | tail gas | tail gas | tail gas | tail gas | tail gas |
| CCS | No | Yes | Yes | Yes | Yes | Yes | Yes |
| Tailgas | No | No | No | 75% | No | 50% | 75% |
| recycle | | | | | | | |
| WGS | HTS | HTS | HTS | HTS | HTS + LTS | HTS + LTS | HTS + LTS |
| H₂ yield | 0.29 | 0.28 | 0.34 | 0.40 | 0.38 | 0.41 | 0.44 |
| (kgH₂/ | | | | | | | |
| kgCH₄) | | | | | | | |
| Energy | 81% | 78% | 86% | 83% | 95% | 91% | 89% |
| efficiency | | | | | | | |
| CCS yield | — | 56% | 66% | 91% | 87% | 93% | 96% |
| (% of | | | | | | | |
| total C) | | | | | | | |
| CO₂ | — | 46% | 28% | 7% | 10% | 5% | 2% |
| emissions | | | | | | | |
| relative to | | | | | | | |
| base case | | | | | | | |
| 1 (per MJ | | | | | | | |
| H₂) | | | | | | | |

Referring to Table 1, using low-carbon electricity to provide heat for SMR instead of using fuel gas increases the hydrogen yield, increases energy efficiency, increases CCS yield, and reduces the carbon dioxide emissions relative to base case 2, in each configuration. Configurations that include low temperature shift reactors provide impressive energy efficiencies. Configurations that include recycling of the tail gas provide impressive hydrogen yields and facilitate surprisingly high CCS yields (e.g., greater than 90%). In addition, when comparing the incremental hydrogen produced per unit feed natural gas (or biogas) with the additional net electric power required we find that this methodology produces 60-200% more hydrogen than if the same amount of power had been used to produce hydrogen via electrolysis.

Advantageously, the high CCS yields (e.g., greater than 90%) are achieved without having to capture carbon dioxide from flue gas, which is more challenging due to the low partial pressure of carbon dioxide and/or the presence of nitrogen. Accordingly, the CCS process may be less expensive. Providing high CCS yields is particularly advantageous when the feedstock includes biomethane, which can drive the carbon intensity of the hydrogen below zero.

Further advantageously, the high energy efficiencies (e.g., greater than 83%) surpass those commonly reported for either conventional SMR with CCS and/or green hydrogen. The energy efficiency for conventional SMR is typically less than about 81% (e.g., as shown in Table 1). The energy efficiency for green hydrogen is often reported to be between about 60% and 80%, depending on the technology. These relatively low energy efficiencies are often seen as drawbacks and/or are associated with excessive heat. Accordingly, certain embodiments of the disclosure can produce hydrogen using low-carbon electricity with higher energy efficiency.

Yet further advantageously, the high hydrogen yields mean that more hydrogen can be produced from a given amount of feedstock (i.e., relative to conventional SMR). This is particularly advantageous when the feedstock includes relatively scarce renewable feedstocks. Moreover, the processes of Examples 1-5 can produce more hydrogen (e.g., 50% to 100% more) than electrolysis for a given quantity of electricity, while also capturing carbon dioxide. While green hydrogen may not produce significant carbon emissions, it does not remove carbon from the atmosphere. In contrast, when hydrogen is produced according to Examples 1-5 using a biomethane feedstock, the carbon intensity of the resulting hydrogen can be negative.

The terminology used herein is for the purpose of describing certain embodiments only and is not intended to be limiting of the invention. For example, as used herein, the singular forms “a,” “an,” and “the” may include plural references unless the context clearly dictates otherwise. The terms “comprises”, “comprising”, “including”, and/or “includes”, as used herein, are intended to mean “including but not limited to.” The term “and/or”, as used herein, is intended to refer to either or both of the elements so conjoined. The phrase “at least one” in reference to a list of one or more elements, is intended to refer to at least one element selected from any one or more of the elements in the list of elements, but not necessarily including at least one of each and every element specifically listed within the list of elements. Thus, as a non-limiting example, the phrase “at least one of A and B” may refer to at least one A with no B present, at least one B with no A present, or at least one A and at least one B in combination. In the context of describing the combining of components by the “addition” or “adding” of one component to another, or the separating of components by the “removal” or “removing” of one component from another, those skilled in the art will understand that the order of addition/removal is not critical (unless stated otherwise). The terms “remove”, “removing”, and “removal”, with reference to one or more impurities, contaminants, and/or constituents of biogas, includes partial removal. The terms “cause” or “causing”, as used herein, may include arranging or bringing about a specific result (e.g., a withdrawal of a gas), either directly or indirectly, or to play a role in a series of activities through commercial arrangements such as a written agreement, verbal agreement, or contract. The term “associated with”, as used herein with reference to two elements (e.g., a fuel credit associated with the transportation fuel), is intended to refer to the two elements being connected with each other, linked to each other, related in some way, dependent upon each other in some way, and/or in some relationship with each other. The terms “first”, “second”, etc., may be used to distinguish one element from another, and these elements should not be limited by these terms. The term “plurality”, as used herein, refers to two or more. The term “providing” as used herein with respect to an element, refers to directly or indirectly obtaining the element and/or making the element available for use. The terms “upstream” and “downstream”, as used herein, refer to the disposition of a step/stage in the process with respect to the disposition of other steps/stages of the process. For example, the term upstream can be used to describe a step/stage that occurs at an earlier point of the process, whereas the term downstream can be used to describe a step/stage that occurs later in the process. Unless defined otherwise, all technical and scientific terms used herein have the same meanings as commonly understood by one of ordinary skill in the art.

Of course, the above embodiments have been provided as examples only. It will be appreciated by those of ordinary skill in the art that various modifications, alternate configurations, and/or equivalents will be employed without departing from the scope of the invention. Accordingly, the scope of the invention is therefore intended to be limited solely by the scope of the appended claims.

1. A process of producing fuel, fuel intermediate, chemical product, or any combination thereof, the process comprising:
providing biomethane, the biomethane produced from a biomethane production process comprising converting biomass to biomethane;
generating hydrogen in a hydrogen production process comprising:
(a) producing syngas in a process comprising subjecting feed comprising the biomethane provided to methane reforming, the methane reforming conducted in one or more reactors, at least one of the one or more reactors configured to provide electrically generated heat for the methane reforming, the heat for the methane reforming at least partially produced using low-carbon electricity, and,
(b) subjecting the syngas to a purification process wherein hydrogen is separated from at least carbon dioxide, thereby producing a stream enriched in hydrogen;
wherein carbon-containing material derived from the biomass is stored and/or used in at least one carbon capture and storage process, wherein the carbon-containing material comprises:
(i) carbon dioxide produced from the hydrogen production process, and
(ii) carbon dioxide produced from the biomethane production process; and
wherein inputs, outputs, or a combination thereof from each of the biomethane production process, the hydrogen production process, and the at least one carbon capture and storage process are monitored, wherein each of the inputs is a material input or energy input and wherein each of the outputs is a material output or an energy output.

2. The process according to claim 1, wherein the biomethane production process comprises subjecting the biomass to anaerobic digestion, the anaerobic digestion producing biogas and digestate, the biomethane production process further comprising subjecting the biogas to biogas upgrading, wherein the carbon dioxide produced from the biomethane production process in (ii) includes carbon dioxide from the biogas.

3. The process according to claim 2, wherein the carbon-containing material further comprises (iii) residue from the biomethane production process or (iv) carbon-containing material derived from the residue, and wherein the residue comprises at least part of the digestate.

4. The process according to claim 3, wherein the carbon-containing material comprises (iv) carbon-containing material derived from the residue, and wherein the carbon-containing material derived from the residue is produced from a process comprising subjecting at least part of the digestate to combustion, pyrolysis, gasification, hydrothermal treatment, or wet oxidation.

5. The process according to claim 1, wherein the carbon-containing material further comprises (iii) residue from the biomethane production process or (iv) carbon-containing material derived from the residue.

6. The process according to claim 5, wherein the carbon-containing material derived from the residue comprises carbon dioxide, biochar, tar, biooil, carbonate, or any combination thereof.

7-10. (canceled)

11. The process according to claim 1, wherein the electrically generated heat is produced using renewable electricity.

12. The process according to claim 1, further comprising providing the stream enriched in hydrogen for use as fuel.

13. The process according to claim 1, further comprising providing the stream enriched in hydrogen for use as a feedstock for a production process, wherein the production process produces at least one fuel, at least one fuel intermediate, at least one chemical product, or any combination thereof.

14. The process according to claim 13, wherein the stream enriched in hydrogen is used as a feedstock to produce a fuel, and wherein the production process comprises hydrogenating crude-oil derived liquid hydrocarbon with the hydrogen.

15. The process according to claim 14, wherein the stream enriched in hydrogen is used as feedstock for ammonia production.

16. The process according to claim 1, wherein hydrogen from the stream enriched in hydrogen has a carbon intensity that is less than 0 gCO₂e/MJ, wherein the biomethane has a carbon intensity that is less than 0 gCO₂e/MJ, or a combination thereof.

17. The process according to claim 1, wherein the biomass comprises manure, agricultural residue, energy crop, or any combination thereof.

18. The process according to claim 1, wherein the feed comprises natural gas.

19. The process according to claim 1, wherein the at least one of the one or more reactors is configured to provide heat for the methane reforming directly using resistive heating.

20. The process according to claim 1, wherein the at least one of the one or more reactors is configured to provide heat for the methane reforming using a heat transfer fluid.

21. The process according to claim 20, wherein the heat transfer fluid comprises a gas.

22. The process according to claim 20, wherein the at least one of the one or more reactors is configured to provide heat for the methane reforming using a heat storage medium.

23. The process according to claim 22, wherein the heat storage medium comprises particulate solid material or molten salt.

24. The process according to claim 1, wherein the production of hydrogen in the process achieves a percentage reduction in lifecycle greenhouse gas emissions compared to a baseline hydrogen production that is at least 40%, where the baseline hydrogen production refers to hydrogen production by steam methane reforming where high-temperature steam is used to produce hydrogen from fossil-based natural gas and where there is no carbon capture and storage.

25. The process according to claim 1, wherein the purification process comprises an amine-based absorption process and an adsorption process, and wherein at least part of a tail gas from the adsorption process is recycled such that it is subjected to the methane reforming.

26. The process according to claim 1, wherein the purification process comprises pressure swing adsorption, and wherein at least part of a tail gas from the pressure swing adsorption is recycled such that it is subjected to the methane reforming.

27. A process of producing hydrogen, the process comprising:
providing biomethane, the biomethane produced from a biomethane production process comprising anaerobic digestion of biomass, the anaerobic digestion producing biogas and digestate, the biomethane used in a hydrogen production process comprising:
(a) producing syngas in a process comprising subjecting a feed comprising the biomethane to methane reforming, the methane reforming conducted in one or more reactors, at least one of the one or more reactors configured to provide electrically generated heat for the methane reforming, the heat for the methane reforming at least partially produced using low-carbon electricity, and,
(b) subjecting the syngas to a purification process wherein hydrogen is separated from at least carbon dioxide, thereby producing a stream enriched in hydrogen;
wherein carbon-containing material derived from the biomass is stored and/or used in at least one carbon capture and storage process, wherein the carbon-containing material comprises:
(i) carbon dioxide produced from the hydrogen production process,
(ii) carbon dioxide from the biogas, and
(iii) carbon-containing material produced from processing at least part of the digestate.

28. The process according to claim 27, wherein the carbon-containing material in (iii) comprises carbon dioxide.

29. The process according to claim 27, wherein the methane reforming is configured such that hydrogen is produced with greater than 82.5% energy efficiency.
